(19) United States
(12) Patent Application Publication
(10) Pub. No.: US 2024/0277523 A1
(43) Pub. Date: Aug. 22, 2024

(54) ADJUSTABLE SHUNTING SYSTEMS WITH CONTROL ELEMENTS, AND ASSOCIATED SYSTEMS AND METHODS

(71) Applicant: Shifamed Holdings, LLC, Campbell, CA (US)

(72) Inventors: Eric Schultz, Los Altos, CA (US); Robert Chang, Belmont, CA (US); Katherine Sapozhnikov, Campbell, CA (US); Tom Saul, Moss Beach, CA (US); Richard Lilly, San Jose, CA (US); Michael Drews, Palo Alto, CA (US); Tessa Bronez, Campbell, CA (US)

(21) Appl. No.: 18/572,101

(22) PCT Filed: Jun. 28, 2022
(86) PCT No.: PCT/US2022/035324

§ 371 Date: Dec. 19, 2023

Related U.S. Application Data
(60) Provisional application No. 63/215,633, filed on Jun. 28, 2021.

(51) Int. Cl. A61F 9/007

(52) U.S. Cl. CPC A61F 9/00781; A61F 2210/0023; A61F 2240/001; A61F 2250/0014; A61F 2250/0047; A61F 2250/0069; A61F 2310/00023

(57) ABSTRACT

The present technology is generally directed to adjustable shunting systems for draining fluid from a first body region to a second body region. The adjustable shunting systems include an actuation assembly for controlling the flow of fluid through the system. For example, the actuation assembly can include one or more fluid inlets in fluid communication with an environment external to the system. The actuation assembly can further include one or more actuators configured to move a corresponding control element to control the flow of fluid through the fluid inlets. The actuator can also have a first actuation element and a second actuation element configured to move the control element between a first position in which the control element substantially prevents fluid flow through the corresponding inlet and a second position in which the control element does not substantially prevent fluid flow through the fluid inlets.

### CROSS-REFERENCE TO RELATED APPLICATION(S)

This application claims the benefit of U.S. Provisional Patent Application No. 63/215,633, filed Jun. 28, 2021, and incorporated by reference herein in its entirety.

### TECHNICAL FIELD

The present technology generally relates to implantable medical devices and, in particular, to adjustable shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. The flow of fluid through the shunting systems is primarily controlled by the pressure gradient across the shunt and the physical characteristics of the flow path defined through the shunt (e.g., the resistance of the shunt lumen(s)). However, most shunting systems have a single static flow path that is not adjustable. Accordingly, one challenge with conventional shunting systems is selecting the appropriate size shunt for a particular patient. A shunt that is too small may not provide enough therapy to the patient, while a shunt that is too large may create new issues in the patient. Despite this, most conventional shunts cannot be adjusted after implantation and, therefore, cannot be adjusted or titrated to meet the patient's individual and variable needs and/or to account for changes in flow-related characteristics, such as flow volume, inflow pressure, and/or outflow resistance.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A-ID illustrate an intraocular shunting system configured in accordance with select embodiments of the present technology.

FIGS. 2A-2C illustrate select aspects of the actuation assembly of FIG. 1C with other aspects of the system omitted for clarity.

FIGS. 3A and 3B illustrate an actuation assembly configured in accordance with select embodiments of the present technology.

FIGS. 4A-4C illustrate another actuation assembly configured in accordance with select embodiments of the present technology.

FIG. 4D is a block diagram of a method for manufacturing an actuation assembly in accordance with select embodiments of the present technology.

FIGS. 5A-5C illustrate another actuation assembly configured in accordance with select embodiments of the present technology.

FIGS. 6A and 6B illustrate a first actuator of the actuation assembly shown in FIG. 5C with certain aspects of the actuation assembly omitted for clarity.

FIGS. 7A and 7B illustrate a first actuator of the actuation assembly shown in FIGS. 6A and 6B, with certain aspects of the first actuator omitted for clarity.

FIGS. 8A and 8B are top views of an actuation assembly configured in accordance with embodiments of the present technology.

FIGS. 9A and 9B are top views of an actuation assembly configured in accordance with further embodiments of the present technology.

### DETAILED DESCRIPTION

The present technology is generally directed to adjustable shunting systems for draining fluid from a first body region to a second body region. The adjustable shunting systems include an actuation assembly for controlling the flow of fluid through the system. For example, the actuation assembly can include one or more fluid inlets in fluid communication with an environment external to the system. The actuation assembly can further include one or more actuators configured to control the flow of fluid through the fluid inlets. In particular, each actuator can include a control element corresponding to and configured to interface with one of the fluid inlets. For example, each control element can be vertically or axially aligned with a corresponding fluid inlet. The actuator can also have a first actuation element and a second actuation element configured to move the control element between (a) a first position in which the control element substantially prevents fluid flow through the corresponding inlet (e.g., the control element covers or blocks the inlet) and (b) a second position in which the control element does not substantially prevent fluid flow through the corresponding fluid inlet (e.g., the fluid inlet is accessible).

As described in greater detail below, it is expected that in at least some embodiments the present technology may exhibit one or more advantageous characteristics that improve operation of adjustable shunting systems. For example, at least some of the actuation assemblies are expected to exhibit improved thermal isolation between the first and second actuation elements to reduce unintentional heating of an un-actuated/non-targeted actuation element. Additionally, at least some of the actuation assemblies are expected to exhibit improved fluid sealing performance between the control element and the fluid inlet when the control element is in a “closed” position, e.g., due at least in part to the orientation and/or motion of the control elements relative to the fluid inlets. In at least some embodiments, the actuation assemblies can include one or more sealing elements, such as gaskets or elastomeric seals, positioned between the control element and the fluid inlet. These sealing elements are also expected to improve the fluid sealing performance of the actuation assemblies. Furthermore, at least some of the actuation assemblies are expected to exhibit improved manufacturing characteristics, e.g., such that multiple actuators can be produced simultaneously and/or be automatically deformed relative to a preferred and/or original geometry during the assembly process. Of course, the present technology may also provide additional advantageous characteristics not expressly described herein.

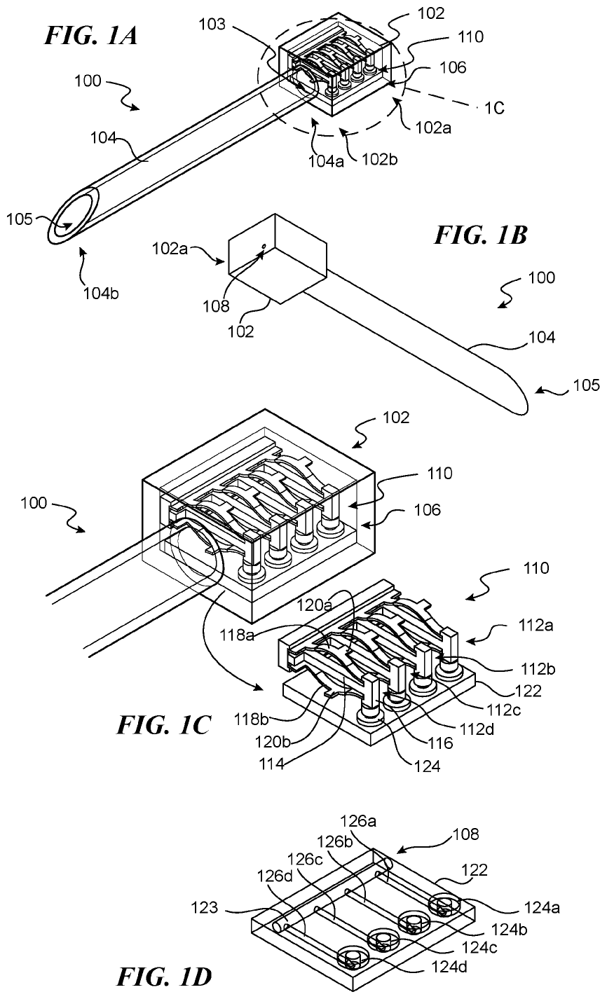

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-10.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1D illustrate an intraocular shunting system (“the system 100”) configured in accordance with select embodiments of the present technology. In particular, FIG. 1A is a perspective view of the system 100, FIG. 1B is another perspective view of the system 100, FIG. 1C is a perspective view of the region marked as “1C” in FIG. 1A and further includes a view of a shape memory actuation assembly 110 (“the actuation assembly 110”) of the system 100 with other aspects of the system 100 omitted for clarity, and FIG. 1D is a perspective view of a base 122 of the actuation assembly 110. As described in greater detail below, the system 100 is configured to provide an adjustable therapy for draining fluid from a first body region, such as to drain aqueous from an anterior chamber of a patient's eye.

Referring first to FIG. 1A, the system 100 includes a housing 102 and a generally elongate drainage element 104 (“the drainage element 104”). The housing 102 has a first end portion 102a and a second end portion 102b, and defines a chamber 106, which, as described below, is configured to receive and house an actuation assembly 110. The drainage element 104 can have a hollow interior or channel 105 extending between a first end portion 104a and a second end portion 104b. The chamber 106 and the channel 105 can be fluidly connected to each other to facilitate drainage of fluid from within the chamber 106 via the channel 105. For example, in the illustrated embodiment the second end portion 102b of the housing 102 further includes an opening or port 103 that fluidly couples the chamber 106 to the first end portion 104a of the drainage element 104 and the channel 105.

The housing 102 and drainage element 104 can be composed of a same or different material. In some embodiments, the housing 102 and/or drainage element 104 are composed of a slightly elastic or flexible biocompatible material (e.g., silicone, etc.). Although the housing 102 is depicted as a rectangular prism in FIGS. 1A and 1B, in other embodiments the housing 102 can be, for example, a cylinder, a triangular prism, a square prism, a pentagonal prism, a cone, a pyramid, or any other suitable shape. Similarly, although the drainage element 104 is depicted as having a circular cross-sectional shape in FIGS. 1A and 1B, in other embodiments the drainage element 104 can have a cross-sectional shape that is, for example, ovular, triangular, square, pentagonal, hexagonal, or any other suitable shape.

Referring next to FIG. 1B, the first end portion 102a of the housing 102 further includes a housing inlet 108 that permits fluid to enter the housing 102. As described below with respect to FIGS. 1C and 1D, the fluid entering the housing 102 via the housing inlet 108 can be selectively permitted to flow into the chamber 106. Once the fluid is in the chamber 106, it can drain via the channel 105. For example, in some embodiments, the housing 102 is positioned at least partially within a first body region (e.g., an anterior chamber of a patient's eye), the second end portion 104b of the drainage element is positioned at least partially in a second body region (e.g., a desired drainage location such as a bleb space), and the housing inlet 108 is configured to allow fluid (e.g., aqueous) to enter the housing 102 and drain from the chamber 106 through the drainage element 104 and into the second body region via the channel 105.

Referring next to FIG. 1C, the amount of fluid that flows through the system 100 can be controlled by the actuation assembly 110. The actuation assembly 110 is positioned with the chamber 106 and includes one or more actuators (e.g., a first actuator 112a, a second actuator 112b, a third actuator 112c, and a fourth actuator 112d; collectively “the actuators 112”). Labels for the features of the first, second, and third actuators 112a-c are omitted in FIG. 1C solely for the purpose of clarity; each of the first, second, and third actuators 112a-c can be configured generally similar or the same as the fourth actuator 112d. For example, each of the actuators 112 can include a generally elongate actuator body portion 114 (“the actuator body 114”) and a control element 116 configured to moveably interface with a corresponding opening 124 (e.g., a fluid inlet, hereinafter referred to as “fluid inlet 124”), e.g., to move between a first (e.g., open) position in which the control element 116 does not substantially prevent fluid from flowing through the fluid inlet 124 and a second (e.g., closed) position in which the control element 116 substantially prevents fluid from flowing though the fluid inlet 124. In some embodiments, the control element 116 can be configured to move between one or more intermediate positions between the first position and the second position. Movement of the control element 116 to one or more intermediate positions can facilitate adjustment of fluid flow through the fluid inlet 124 to a rate that is above that of the (e.g., closed) second position, but below that of the (e.g., fully open) first position. In some embodiments, the actuator body 114 is contiguous with the control element 116 to form a unitary structure.

Each of the actuators 112 can further include a first (e.g., upper) actuation element 118a and a second (e.g., lower) actuation element 118b (collectively, “the actuation elements 118”) that drive movement of the control element 116 between the first position and the second position. The first actuation element 118a and the second actuation element 118b can be composed, at least partially, of a shape memory material or alloy (e.g., Nitinol). Accordingly, the first actuation element 118a and the second actuation element 118b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In relation to the second material state, the first material state possesses reduced mechanical properties (e.g., Young's modulus) that cause bodies in the first material state to be more easily deformable (e.g., compressible, expandable, etc.) with respect to the second material state. In the second material state, the first actuation element 118a and the second actuation element 118b may have increased mechanical properties causing an increased preference toward a specific preferred geometry (e.g., original geometry, manufactured or fabricated geometry, heat set geometry, etc.). The first actuation element 118a and the second actuation element 118b can be selectively and independently transitioned between the first material state and the second material state by applying energy (e.g., heat) to the first actuation element 118a or the second actuation element 118b to heat it above a transition temperature (e.g., an austenite finish temperature). If the first actuation element 118a (or the second actuation element 118b) is deformed relative to its preferred geometry when heated above the transition temperature, the first actuation element 118a (or the second actuation element 118b) will transition toward and/or to its preferred geometry.

The first actuation element 118a and the second actuation element 118b generally act in opposition. For example, the first actuation element 118a can be actuated to move the control element 116 toward and/or to the second position, and the second actuation element 118b can be actuated to move the control element 116 toward and/or to the first position. Additionally, the first actuation element 118a and the second actuation element 118b move in concert with each other such that as one transitions toward its preferred geometry upon material phase transition, the other is deformed relative to its preferred geometry. This enables the actuation elements to be repeatedly actuated and the control element 116 to be repeatedly cycled between the first position and the second position. Additional details regarding the operation of shape memory actuators, as well as adjustable glaucoma shunts, are described in U.S. Pat. Nos. 11,291,585, 11,166,849, and International Patent Application Nos. PCT/US20/55144, PCT/US20/55141, PCT/US21/14774, PCT/US21/18601, PCT/US21/23238, and PCT/US21/27742, the disclosures of which are all incorporated by reference herein in their entireties and for all purposes.

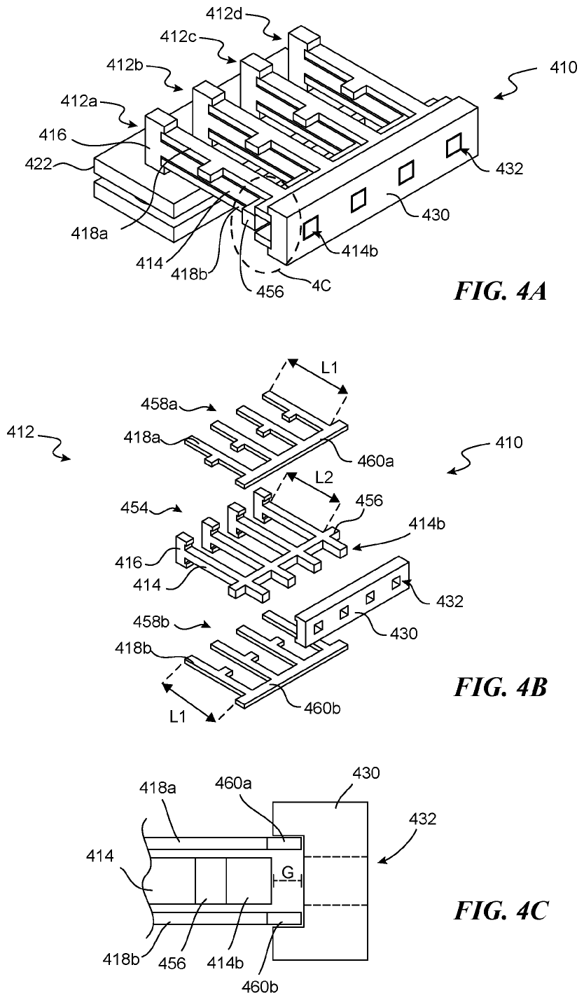

In some embodiments, the actuation elements 118 can be “stressed,” “strained,” “loaded,” or deformed relative to their preferred geometries prior to use in a system, such as the system 100. For example, the first and second actuation elements 118a-b can be deformed contemporaneously or substantially simultaneously from their preferred geometry to a same or generally similar deformed (e.g., stressed, tensioned, compressed, etc.) geometry, e.g., so that the first and second actuation elements 118a-b can be actuated to move the actuator between the first and second positions as described previously. The stressing of the actuation elements is discussed in greater detail below with reference to FIGS. 4A-4C.

The first actuation element 118a can include a first tab or target 120a, and the second actuation element 118b can include a second tab or target 120b (collectively, “the targets 120”). The targets 120 can extend (e.g., laterally, horizontally, etc.) from the respective first and second actuation elements 118a-b. For example, the first target 120a can extend in a first direction from the first actuation element 118a and the second target 120b can extend in a second direction from the second actuation element 118b that is different (e.g., opposite) than the first direction. Because the targets 120 extend relative to the actuation elements 118 in different directions, the targets 120 are not aligned along a common (e.g., vertical) axis (e.g., the first target 120a is not “stacked” on top of the second target 120b) even though the actuation elements 118a-b are arranged along a common (e.g., vertical) axis (e.g., the first actuation element 118a is “stacked” on top of the second actuation element 118b). Both targets 120 are therefore expected to be accessible to energy (e.g., laser energy), even if the body of one of the actuation elements 118 is generally not directly accessible. The targets 120 can therefore be used to selectively and independently actuate the first and second actuation elements 118a-b (e.g., selectively heating the first or second actuation element 118a-b to transition it from the first material state to the second material state). For example, to actuate the first actuation element 118a, heat/energy can be applied to the first target 120a, such as from an energy source positioned external to the patient's eye (e.g., a laser). The heat applied to the first target 120a spreads through at least a portion of the first actuation element 118a, which can heat the first actuation element 118a above its transition temperature. To actuate the second actuation element 118b heat/energy can be applied to the second target 120b. The heat applied to the second target 120b spreads through the second actuation element 118b, which can heat at least the portion of the second actuation element 118b above its transition temperature.

In some embodiments, the first and second actuation elements 118a-b are at least partially thermally and/or energetically isolated from each other, e.g., to prevent or substantially limit energy applied to a target actuation element from spreading to a non-targeted actuation element. Energy that spreads to a non-targeted actuation element can at least partially heat the non-targeted actuation element, which may inadvertently induce a geometric change in the non-targeted actuation element by causing the non-targeted actuation element to transition toward its preferred geometry. This is disadvantageous because the target actuation element generally works in opposition with the non-targeted actuation element, so any shape-memory-based geometric change of the non-targeted actuation element can affect the desired adjustment of the system 100, thereby reducing control of fluid flow through the system 100. Accordingly, it is expected that the actuation assemblies described herein, and/or one or more features thereof, will exhibit improved energetic (e.g., thermal) isolation characteristics of the actuation elements, which can advantageously improve the control of fluid flow through the system 100. For example, in the illustrated embodiment the first and second actuation elements 118a-b are positioned on opposite sides of the actuator body 114. The actuator body 114 and/or the control element 116 can be composed of a material that is at least partially insulating. For example, the control element 116 can be composed of ceramic, carbon, glass, high molecular weight polymers (e.g., Polyethylene Terephthalate (PET)), etc., and/or a material having a relatively low thermal conductivity and/or heat capacity, such as a thermal conductivity and/or a heat capacity that is less than that of the actuation elements 118). In some embodiments, the control element 116 may contain one or more coatings or layers (e.g., oxide, ceramic, carbon, glass, high molecular weight polymers, or other materials with low thermal conductivity), and/or have a high thermal mass (e.g., energy density), to reduce and/or prevent energy (e.g., heat) applied to a target actuation element from spreading to the non-targeted actuation element. In at least some embodiments, the material composing the actuator body 114 and/or the control element 116 can have a mass sufficient to dissipate energy (e.g., heat) transferred from the first and/or second actuation elements 118a-b to the actuator body 114 and/or the control element 116, so as to reduce and/or prevent heat transfer from the target actuation element to the non-targeted actuation element. In some embodiments, the first actuation element 118a and the second actuation element 118b can be separated by a gap (e.g., not physically coupled) to reduce and/or prevent heat transfer from the target actuation element to the non-targeted actuation element.

In embodiments in which some energy does indeed spread from the targeted actuation element to the other actuation elements, it is expected that the energy (e.g., heat) that spreads will have reduced intensity (e.g., temperature) by virtue of the insulating actuator body 114 and thus will not cause substantial heating of these other actuation elements relative to the target actuation element that directly receives the energy. Additionally, in some embodiments each of the first actuation elements 118a can be insulated (e.g., thermally) from each other, and each of the second actuation elements 118b can be insulated (e.g., thermally) from each other. For example, each of the first actuation elements 118a can be coupled to each other by an insulating material (e.g., a material having low thermal conductivity), and each of the second actuation elements 118b can be similarly insulated. In at least some embodiments, the insulating material coupling the first and second actuation elements 118a-b can have a mass sufficient to induce the dissipation of energy, as discussed previously.

In some embodiments, the actuator body 114 can also be stiffer or more rigid than the first and second actuation elements 118a-b, e.g., at least relative to the stiffness of the first and second actuation elements 118a-b in the first material state. For example, the actuator body 114 can be formed from a material having a stiffness greater than the stiffness of the first and second actuation elements 118a-b, and/or the geometry (e.g., width, thickness, etc.) of the actuator body 114 can be configured (e.g., wider, thicker, etc.) such that the actuator body 114 exhibits greater stiffness than the first and second actuation elements 118a-b. This is expected to improve the consistency and/or magnitude of motion of the actuator body 114 and control element 116. For example, this enables the actuation elements 118 to be initially deformed relative to the preferred geometries without substantially deforming the actuator body 114 and control element 116. This also enables the actuator body 114 and control element 116 to have a consistent motion upon actuation of the actuation elements 118. Accordingly, the actuator body 114 can be formed from a material having a stiffness greater than the actuation elements 118. In at least some embodiments, the actuator body 114 can be both insulating and have an increased stiffness relative to the actuation elements.

The actuation assembly 110 further includes the base 122 (e.g., a base plate). Referring next to FIG. 1D, the base 122 can include one or more fluid inlets and/or apertures (e.g., a first fluid inlet 124a, a second fluid inlet 124b, a third fluid inlet 124c, and a fourth fluid inlet 124d; collectively “the fluid inlets 124”) The fluid inlets 124 can be fluidly coupled to the housing inlet 108. For example, each of the fluid inlets 124 is connected to a fluid collection lumen 123 that receives fluid via the housing inlet 108 by a corresponding channel (e.g., the first fluid inlet 124a by a first channel 126a, the second fluid inlet 124b by a first channel 126b, the third fluid inlet 124c by a third channel 126c, and the fourth fluid inlet 124d by a fourth channel 126d; collectively “the channels 126”) to permit fluid to enter the chamber 106 (for the purpose of clarity, chamber 106 is not shown in FIG. 1D) from an environment external to the system 100. Fluid that enters the housing inlet 108 can pass through the channels 126 and the corresponding fluid inlet 124 to enter the chamber 106.

Each of the actuators 112 is configured to control the flow of fluid through a corresponding fluid inlet 124. For example, the first actuator 112a is configured to control the flow of fluid through the first fluid inlet 124a, the second actuator 112b is configured to control the flow of fluid through the second fluid inlet 124b, the third actuator 112c is configured to control the flow of fluid through the third fluid inlet 124c, and the fourth actuator 112d is configured to control the flow of fluid through the fourth fluid inlet 124d. In the first position, the control element 116 of each of the actuators 112 does not substantially prevent and/or interfere with fluid flow through the corresponding fluid inlet 124. In the second position, the control element 116 of each of the actuators 112 can form a fluid seal with the corresponding fluid inlet 124, e.g., so as to substantially prevent or otherwise interfere with fluid flow through the fluid inlet 124. In some embodiments, the control element 116 of the actuators does not form a complete fluid seal in the second position, but rather permits, for a given pressure, a leakage flow rate, e.g., to ensure that at least some flow through the system 100 is maintained even when the control elements 116 are in the second position.

In operation, the system 100 can be used to drain aqueous from the anterior chamber of the eye to treat glaucoma. Accordingly, when the system 100 is implanted in an eye to treat glaucoma, the first end portion 102a of the housing 102 can be positioned within an anterior chamber of the patient's eye such that the housing inlet 108 is in fluid communication with the anterior chamber, and the second end portion 104b of the drainage element 104 can be positioned in a target outflow location, such as a subconjunctival bleb space, such that the channel 105 is in fluid communication with the target outflow location. As described previously, aqueous can flow into the housing 102 via the housing inlet 108, through the channels 126, the corresponding fluid inlets 124, and the actuation assembly 110 into the chamber 106, and exit via the channel 105. In some embodiments, the orientation of the system 100 can be reversed such that the housing 102 is positioned in a target outflow location and the second end portion 104b is positioned in the anterior chamber.

In some embodiments, the relative level of therapy provided by each of the fluid inlets 124 when unblocked by the corresponding actuator 112 can be the same. In some embodiments, the relative level of therapy provided by each of the fluid inlets 124 when unblocked by the corresponding actuator 112 can be different so that a user may selectively titrate the flow through the system 100 by selectively interfering with or permitting flow through individual fluid inlets 124. For example, under a given pressure, when flow primarily occurs through the first fluid inlet 124a, the system 100 can provide a first drainage rate, when flow primarily occurs through the second fluid inlet 124b, the system 100 can provide a second drainage rate less than the first drainage rate, when flow primarily occurs through the third fluid inlet 124c, the system 100 can provide third drainage rate less than the second drainage rate, and when flow primarily occurs through the fourth fluid inlet 124d, the system 100 can provide fourth drainage rate less than the third drainage rate. The foregoing difference in drainage rates can be achieved based on the different fluid resistance of the channels 126a-d receiving fluid from the respective fluid inlets 124a-d. In some embodiments, the channels 126 can have varied widths and/or lengths that result in varied fluid resistances. Although the channels 126 illustrated in FIG. 1D are configured in parallel, in other embodiments the channels 126 can be configured in series, for example, as described in International Patent Application No. PCT/US21/14774, previously incorporated by reference herein.

Although depicted as having four actuators 112a-d and four fluid inlets 124a-d in FIGS. 1C-1D, in other embodiments the actuation assembly 110 can include more or fewer actuators 112 and fluid inlets 124. For example, the actuation assembly 110 can include one, two, three, five, six, seven, eight, or more actuators 112 and fluid inlets 124.

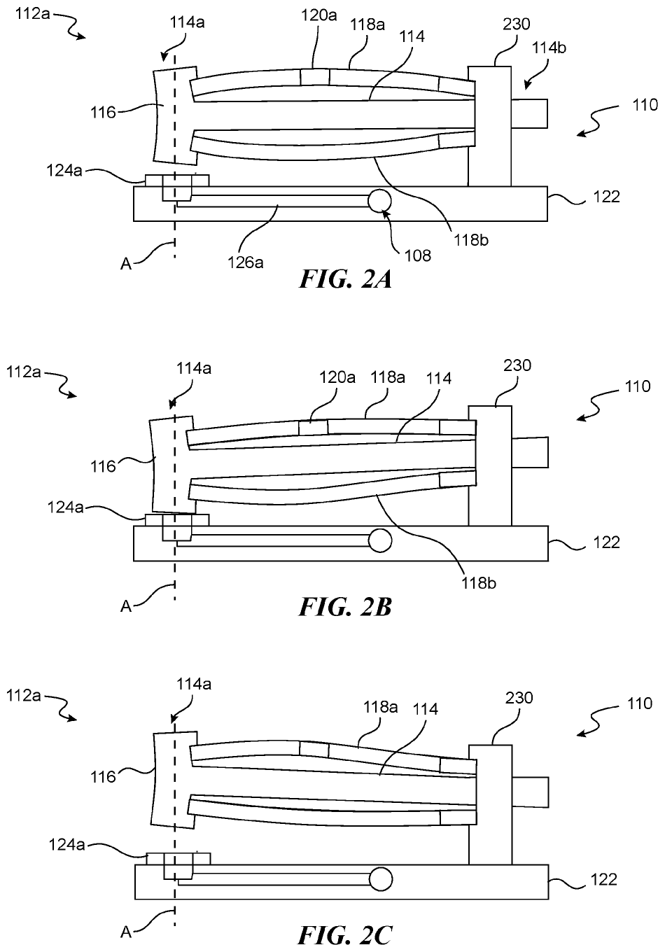

FIGS. 2A-2C illustrate the actuation assembly 110 of FIG. 1C with other aspects of the system 100 described above with reference to FIGS. 1A-1D omitted for clarity. In particular, FIG. 2A is a side view of the first actuator 112a in a non-actuated (post-assembly, stressed, strained, loaded, compressed, etc.) position, FIG. 2B is a side view of the first actuator 112a in the second (e.g., closed) position described with respect to FIGS. 1A-1C, and FIG. 2C is a side view of the first actuator 112a in the first (e.g., open) position described with respect to FIGS. 1A-1C.

Referring first to FIG. 2A, the actuation assembly 110 further includes a bracket or actuator mount 230 coupled to the base 122. The actuator body 114 includes a first end portion 114a that includes the control element 116, and a second end portion 114b at least partially received (e.g., insertably, releasably, fixedly, etc.) by the actuator mount 230. The first and second actuation elements 118a-b are positioned between and contact the first end portion 114a and/or control element 116 of the actuator body 114 and the actuator mount 230. The interaction between the first and second actuation elements 118a-b, the actuator body 114, and the actuator mount 230 can cause the first actuator 112a to move from the first position toward and/or to the second position. In the as-formed or unactuated position illustrated in FIG. 2A, the first and second actuation elements 118a-b are deformed (e.g., compressed, tensioned, stressed, etc.) equally or at least generally equally. However, as discussed previously, applying energy (e.g., heat) to the first or second actuation elements 118a-b can move the first actuator 112a to a first or second position.

FIG. 2B illustrates the actuator 112a after energy has been applied to the first actuation element 118a (e.g., to the first target 120a) to transition the first actuator 112a toward and/or to the second position. Relative to its configuration in FIG. 2A, the first actuation element 118a has expanded toward its preferred geometry, acting against the actuator mount 230 and first end portion 114a of the actuator body 114 to pivot the actuator body 114 relative to the actuator mount 230, and moving the control element 116 toward (e.g., into contact with) the first fluid inlet 124a. When contacting the first fluid inlet 124a, the control element 116 can substantially prevent fluid flow through the first fluid inlet 124a (e.g., by forming a substantially fluid seal). As the first actuator 112a transitions toward the second position, the second actuation element 118b can be deformed (e.g., compressed) relative to its configuration in FIG. 2A. This can allow the second actuation element 118b to act in opposition to the first actuation element 118a, as discussed previously.

FIG. 2C illustrates the actuator 112a after energy has been applied to the second actuation element 118b (e.g., to the second target 120b; not shown in FIG. 2C for clarity) to transition the first actuator 112a from the second position of FIG. 2B toward and/or to the first position. Relative to its configuration in FIG. 2B, the second actuation element 118b has expanded toward and/or to its preferred geometry, acting against the first end portion 114a of the actuator body 114 to pivot the actuator body 114 relative to the actuator mount 230, and moving the control element 116 away from the first fluid inlet 124a. In the first position, the control element 116 does not substantially prevent fluid flow through the first fluid inlet 124a (e.g., no fluid seal is formed). As the first actuator 112a transitions toward and/or to the first position, the first actuation element 118a can be deformed (e.g., compressed) relative to its configuration in FIG. 2B. This can allow the first actuation element 118a to act in opposition to the second actuation element 118b, as discussed previously.

As shown and described above with respect to FIGS. 2A-2C, the control element 116 is configured to move in a plane that is substantially parallel to a central axis A extending through the first fluid inlet 124a (e.g., as opposed to sliding over the fluid inlet 124a by moving in a plane that is perpendicular to the central axis A extending through the first fluid inlet). For example, the motion of the control element 116 can be vertically or axially aligned with the central axis extending through the first fluid inlet 124a, such that in the second position (illustrated in FIG. 2B) the control element 116 at least partially contacts (e.g., presses against) the first fluid inlet 124a, e.g., to substantially prevent fluid flow through the first inlet 124a. As one skilled in the art will appreciate from the disclosure herein, in some embodiments the control element 116 may move along a slightly arcuate path rather than a fully linear path as it moves between the first and second positions. Such arcuate movement is still considered substantially parallel to the central axis A and vertically/axially aligned for purposes of this disclosure. Without being bound by theory, it is believed that an improved fluid seal is formed when the motion of and/or force applied by the control element is aligned (e.g., vertically, axially, linearly, etc.) with the fluid inlet. Accordingly, it is expected that, in at least some embodiments, the actuation assemblies described herein will exhibit improved fluid sealing performance. This can advantageously improve control of fluid flow through the system 100.

Although described in the context of the first actuator 112a, a description of FIGS. 2A-2C applies equally to the second, third, and fourth actuators 112b-d of FIG. 1C. In some embodiments, one or more of the actuators 112 of FIG. 1C are actuated in concert to achieve a desired fluid flow rate through the actuation assembly 110. Additionally, although the first actuator 112a is described as operating under compression (e.g., first and second actuation elements 118a-b expand towards their preferred geometry when actuated), in other embodiments the first actuator 112a can be configured to operate under tension (e.g., first and second actuation element 118a-b contract or shorten towards their preferred geometry when actuated).

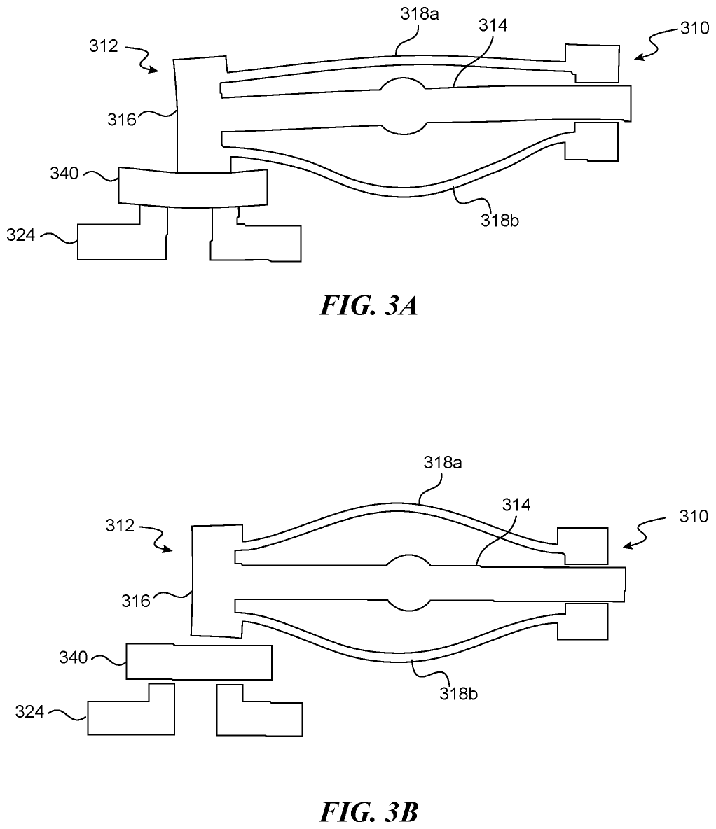

FIGS. 3A and 3B illustrate an actuation assembly 310 configured in accordance with select embodiments of the present technology. The actuation assembly 310 can include elements that are generally similar or the same as the actuation assembly 110 of FIGS. 1A-2C. Accordingly, like numbers are used to designate like elements (e.g., actuator 312 versus first actuator 112a), and the discussion of FIGS. 3A and 3B will be limited to those features that differ from FIGS. 1A-2C and any additional aspects necessary for context. Accordingly, a description of the actuation assembly 310 with respect to FIGS. 3A and 3B applies equally to the actuation assembly 110 of FIGS. 1A-2C.

Referring first to FIG. 3A, the actuation assembly 310 includes an actuator 312 having a first (e.g., upper) actuation element 318a, a second (e.g., lower) actuation element 318b, and a control element 316 aligned (e.g., vertically, axially, linearly etc.) with a fluid inlet 324. The actuation assembly 310 further includes a fluid inlet 324 and a membrane or sealing element 340 positioned between the fluid inlet 324 and the control element 316. The sealing element 340 can be or include an elastomer (e.g., silicone, polymethyl methacrylate (“PMMA”), polydimethylsiloxane (“PDMS”), etc.) or any other suitable material that, when pressed against/into the fluid inlet 324, prevents or reduces fluid from flowing through the inlet 324. When the actuator 312 is in a second (e.g., closed) position such as that illustrated in FIG. 3A, the control element 316 can contact the sealing element 340 such that the sealing element 340 abuts the fluid inlet 324 and substantially prevents or reduces fluid flow through the fluid inlet 324. It is expected that the inclusion of the sealing element 340 can further improve the prevention of fluid flow through the fluid inlet 324.

FIG. 3B shows the actuator 312 in the first (e.g., open) position. In the first position, the control element 316 moves away from the sealing element 340 such that the sealing element 340 does not substantially prevent or reduce fluid flow through the fluid inlet 324. As described previously, the actuator 312 can be used to control the fluid flow through the actuation assembly 310.

FIGS. 4A-4C are views of an actuation assembly 410 configured in accordance with select embodiments of the present technology. The actuation assembly 410 can include elements that are generally similar or the same as the actuation assembly 110 of FIGS. 1A-2C and/or the actuation assembly 310 of FIGS. 3A and 3B. Accordingly, like numbers are used to designate like elements (e.g., first actuator 412a versus actuator 312, first actuator 112a), and the discussion of FIGS. 4A and 4B will be limited to those features that differ from FIGS. 1A-3B and any additional aspects necessary for context. Accordingly, a description of the actuation assembly 410 in FIGS. 4A and 4B applies equally to the actuation assembly 110 of FIGS. 1A-2C and/or the actuation assembly 310 of FIGS. 3A and 3B.

FIG. 4A is a perspective view of the actuation assembly 410 at a stage of a manufacturing process. The actuation assembly 410 includes one or more actuators 412 (e.g., a first actuator 412a, a second actuator 412b, a third actuator 412c, and a fourth actuator 412d). Each of the actuators 412a-d includes a first or upper actuation element 418a, a second or lower actuation element 418b, and an actuator body 414. At least part of a second end portion 414b of the actuator body 414 can be received (e.g., insertably, releasably, fixedly, etc.) by a corresponding aperture 432 in the actuator mount 430. The actuation assembly 410 can further include a base plate 422, and each of the actuators 412 can be positioned above the base plate 422 (e.g., aligned with a fluid inlet; not shown in FIG. 4A for clarity) and/or one or more sealing elements 440 (e.g., which can be the same as or generally similar to the sealing element 340 described above with reference to FIGS. 3A and 3B). At a later stage of the manufacturing process, the actuator mount 430 can be moved toward the control elements 416 to contact an actuator body support 456. As will be described in greater detail below, this can strain the first and second actuation elements 418a-b, e.g., by deforming the first and second actuation elements 418a-b relative to their preferred geometry.

FIG. 4B is an exploded view of the actuators 412 of FIG. 4A with other aspects of the actuation assembly 410 omitted for clarity. The actuators 412 can be formed from one or more sheets/elements that can be manufactured separately. For example, the actuators 412 can be formed from a first or upper sheet 458a, a second or lower sheet 458b, and a third or middle sheet 454. The first sheet 458a can include the one or more first actuation elements 418a coupled to a first actuation element support 460a; the second sheet 458b can include the one or more second actuation elements 418b coupled to a second actuation element support 460b; and the third sheet 454 can include the one or more actuator bodies 414, control elements 416, and end portions 414b coupled to the actuator body support 456. The first sheet 458a, the second sheet 458b, the third sheet 454, and the actuator mount 430 can be configured to be combined (e.g., assembled) in a predetermined configuration and/or order. For example, to assemble the actuation assembly 410, the first and second sheets 458a-b can be positioned on opposite sides of the third sheet 454, and at least partially between the control elements 416 and the actuator mount 430, e.g., as illustrated in FIG. 4A, such that the first and second actuation element supports 460a-b contact (e.g., are received within) the actuator mount 430.

Of note, each of the first and second actuation elements 418a-b can be (e.g., automatically and/or simultaneously) deformed relative to its preferred geometry when coupling the first sheet 458a, the second sheet 458b, and the third sheet 454 to the actuator mount 430. During the assembly process, e.g., as illustrated in FIG. 4A, each of the first and second actuation elements 418a-b are positioned between the control element 416 and that actuator mount 430. For example, before assembly each of the first and second sheets 458a-b can have a first length L1, and the portion of the third sheet 454 that includes the actuator bodies 414 and the actuator body support 456 can have a second length L2 less than the first length L1. Accordingly, when the sheets are stacked as shown in FIG. 4A, the actuation elements 418 extend between and contact both the control elements 416 and the actuator mount 430, whereas the second end portions 414b of the actuator bodies 414 is at least partially spaced apart from the actuator mount 430 by a gap G, as best shown in FIG. 4C, which is an enlarged side view of the portion of the actuation assembly 410 indicated in FIG. 4A. As a result, moving the actuator mount 430 toward the control elements 416 such that the second end portion 414b of the actuator bodies 414 is received within the corresponding apertures 432 of the actuator mount 430, and/or moving the actuator mount 430 toward the actuator body support 456 such that the actuator mount 430 contacts the actuator body support 456, causes each of the first and second actuation elements 418a-b to deform relative to a preferred geometry, e.g., to deform the first and second actuation elements 418a-b (e.g., the actuation elements 418 may compress and bow outwardly relative to the actuator body 414, as shown in FIG. 1C). This can allow the first and second actuation elements 418a-b to be used in opposition to each other, e.g., as part of an actuation assembly and/or system as discussed previously.

Incorporating an actuation assembly such as those described above into adjustable shunting systems is expected to provide several advantages. For example, many of the components required to produce an adjustable shunting system capable of providing a titratable and adjustable therapy are very small and difficult to manufacture using conventional techniques for molding plastic, steel, or other non-transparent materials. In contrast, utilizing the actuation assemblies described herein is expected to reduce the complexity of manufacturing. For example, the sheets of the actuation assembly (e.g., the sheets 454, 458a-b of the actuation assembly 410 of FIG. 4B) can be formed via known techniques for fabricating materials at a relatively high resolution (e.g., about 10 microns or less) and high reproducibility. Additionally, and as described previously, assembling the pre-fabricated sheets into the actuation assembly can stress and/or deform the actuation elements, e.g., so that the actuation elements can be used in opposition to each other to control fluid flow through the actuation assembly, thereby simplifying the manufacturing process.

The present technology further includes methods of manufacturing the actuation assemblies described herein. For example, FIG. 4D is a block diagram of a method 480 for making an actuation assembly in accordance with embodiments of the present technology. The method 480 can begin at step 481 by fabricating a first sheet from a first material. This can include, for example, forming one or more actuation elements in the first sheet, such as in the first sheet 458a of FIG. 4B. The first sheet can be formed out of a shape memory material, such as Nitinol, and may be formed via any suitable process having a relatively high resolution (e.g., 3D printing). The first sheet may be formed with certain features described above, such as targets, actuation element supports, and the like.

The method 480 can continue at step 482 by fabricating a second sheet form the first material. The second sheet can include one or more second actuation elements, such as the second sheet 458b of FIG. 4B. Step 482 can be substantially similar or the same as step 481.

The method 480 can continue at step 483 by forming a third sheet from a second material. The third sheet can include one or more actuator bodies, such as the third sheet 454 of FIG. 4B. The second material can have increased stiffness relative to the first material, and/or have a lower conductivity (e.g., thermal conductivity) relative to the first material.

The method 480 can continue at step 484 by forming an actuator mount from a third material. The actuator mount can include one or more apertures, such as the actuator mount 430 of FIGS. 4A-4B. The third material can be a same or different material as the second material.

The method 480 can continue at step 485 by combining the first sheet, the second sheet, the third sheet, and the actuator mount. Each of the first sheet, second sheet, third sheet, and actuator mount can be configured for combination in a predetermined configuration, e.g., as described previously regarding FIGS. 4A-4B.

The method 480 can continue at step 486 by deforming the one or more first and second actuation elements relative to their preferred geometries. In some embodiments, steps 485 and 486 may be combined (e.g., combining the first sheet, the second sheet, the third sheet, and the actuator mount in a predetermined configuration (e.g., step 485) deforms (e.g., automatically deforms) the first and second actuation elements relative a preferred geometry). As discussed previously, this can stress the first and second actuation elements for use in a system, such as the system 100 of FIGS. 1A-ID.

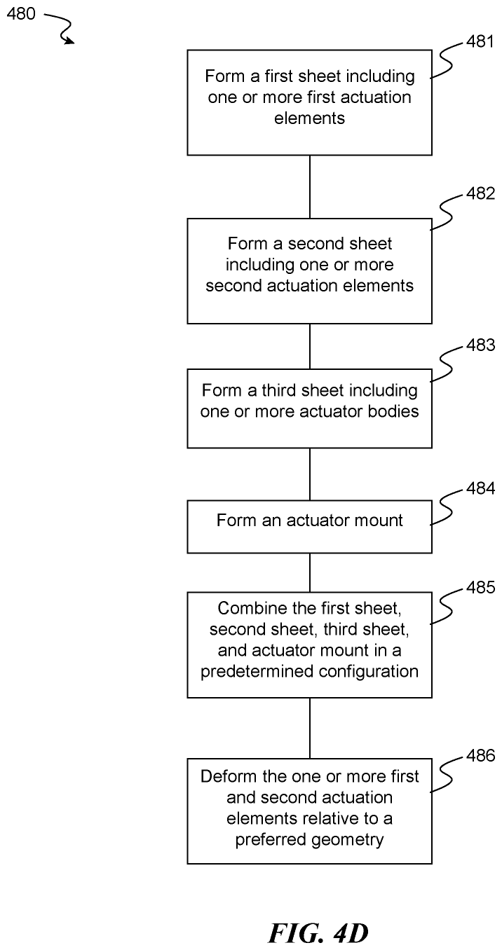

FIGS. 5A-5C are views of an actuation assembly 510 configured in accordance with select embodiments of the present technology. The actuation assembly 510 can include elements that are generally similar or substantially identical to the actuation assembly 110 of FIGS. 1A-2C, the actuation assembly 310 of FIGS. 3A and 3B, and/or the actuation assembly 410 of FIGS. 4A and 4B. Accordingly, like numbers are used to designate like elements (e.g., first actuator 512a versus first actuator 412a, actuator 312, first actuator 112a), and the discussion of FIGS. 5A-5C will be limited to those features that differ from FIGS. 1A-4B and any additional aspects necessary for context. Accordingly, a description of the actuation assembly 510 of FIGS. 5A-5C applies equally to the actuation assembly 110 of FIGS. 1A-2C, the actuation assembly 310 of FIGS. 3A-3B, and/or the actuation assembly 410 of FIGS. 4A and 4B.

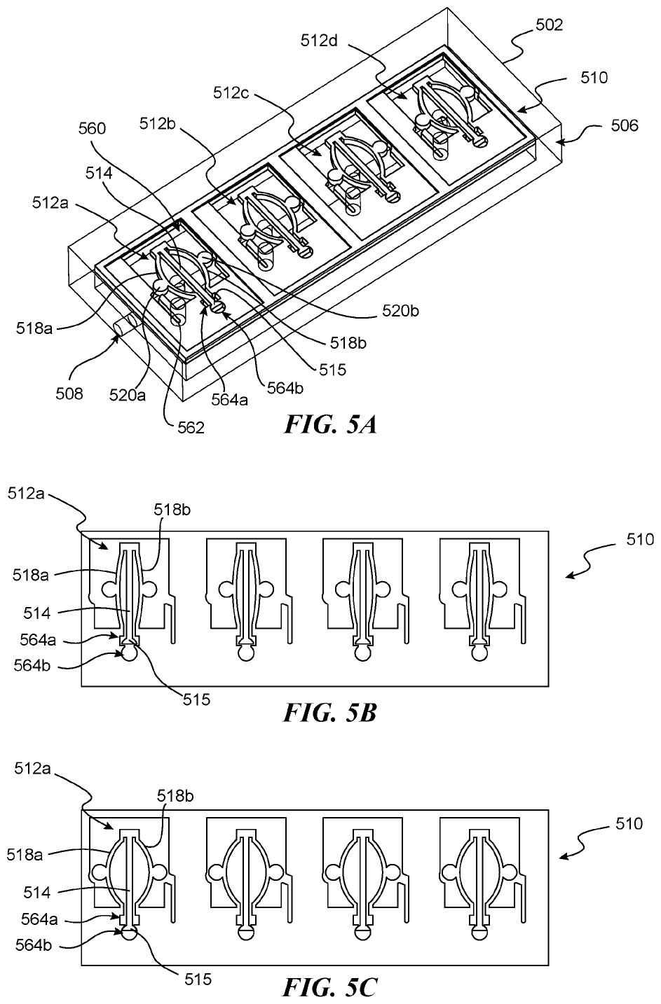

FIG. 5A is a top view of the actuation assembly 510. The actuation assembly 510 can be received and housed by a housing 502, e.g., within a chamber 506 of the housing 502. The housing 502 can be fluidly coupled to an environment outside the housing 502 by a housing inlet 508. The actuation assembly 510 can include one or more actuators (e.g., a first actuator 512a, a second actuator 512b, a third actuator 512c, and a fourth actuator 512d; collectively “the actuators 512”). Each of the actuators 512 includes an actuator body 514, a first actuation element 518a, and a second actuation element 518b. The first actuation element 518a includes a first target 520a, and the second actuation element 518b includes a second target 520b. The first and second targets 520a-b can be positioned at or proximate a midpoint of the respective actuation elements 518a-b. As will be discussed in greater detail below regarding FIGS. 6A-6B, the first and second targets 520a-b can be configured to receive energy (e.g., heat) to actuate the respective first and second actuation elements 518a-b and to control fluid flow through the actuation assembly 510. The actuator body 514 can include a flared end portion 515 having a width greater than the width of the actuator body 514. Although depicted as having a semicircular shape in FIG. 5A, in other embodiments the flared end portion 515 can have other shapes. For example, the flared end portion 515 can be circular, triangular, square, rectangular, etc., or any other suitable shape.

The actuation assembly 510 can further define a plurality of wells 560 corresponding to the actuators 512 such that each of the actuators 512a-d can be positioned within a well 560. Each of the wells 560 can include a well inlet 562 fluidly coupled to the housing inlet 508 such that each of the wells 560 can be fluidly coupled to an environment external to the housing 502. Each of the wells 560 can further include a first chamber 564a and a second chamber 564b. Both the first and second chambers 564a-b can be configured to receive (e.g., insertably, releasably, fixedly, etc.) the flared end portion 515 of the actuator body 514, e.g., such that the flared end portion 515 can be positioned in either the first chamber 564a or the second chamber 564b. For example, the actuation assembly 510 can be manufactured with the flared end portion 515 positioned in the first chamber 564a. As described in greater detail below, moving the flared end portion 515 from the first chamber 564a to the second chamber 564b can cause the first and second actuation elements 518a-b to be deformed (e.g., compressed or extended) relative to their preferred geometry. In some embodiments, the actuation assembly 510 can be a unitary or contiguous structure (e.g., cut from, printed as, or deposited as a single piece of material). For example, each of the actuators 512 can be patterned (e.g., cut, laser cut, formed, etc.) in a single piece of material (e.g., Nitinol), and the wells 560 can correspond to regions of the single piece of material that were removed (e.g., during a subtractive manufacturing process) or where material was not added (e.g., during an additive manufacturing process).

FIGS. 5B and 5C are top views of the actuation assembly 510 of FIG. 5A. In particular, FIG. 5B illustrates the actuation assembly 510 in an “as-formed” and/or “unstressed” configuration, and FIG. 5C illustrates the actuation assembly 510 in a “stressed,” “strained,” “loaded,” and/or “deformed” configuration. Referring first to FIG. 5B, in the pre-stressed configuration the flared end portion 515 is positioned in the first chamber 564a and the first and second actuation elements 518a-b are not substantially deformed relative to their preferred geometry. Referring next to FIG. 5C, the flared end portion 515 has been moved to the second chamber 564b to place the actuation assembly in the stressed configuration. Moving the flared end portion 515 into the second chamber 564b causes the actuation elements 518 to be compressed and bow away from the actuator body 514, thereby deforming the actuation elements 518 relative to their configuration in FIG. 5B. Accordingly, moving the flared end portion 515 from the first chamber 564a to the second chamber 564b can stress the first and second actuation elements 518a-b, e.g., deform the first and second actuation elements 518a-b relative to their preferred geometry such that the first and second actuation elements 518a-b can be used to act in opposition to each other, as described previously. Additionally, and as described in greater detail regarding FIGS. 6A and 6B, the interaction between the flared end portion 515 and the second chamber 564b can allow the actuator body 514 to bend or pivot relative to the second chamber 564b, e.g., to transition from a first (e.g., open) position to a second (e.g., closed) position. Although FIGS. 5B and 5C describe the actuation assembly in a pre-loaded and loaded configuration, respectively, in other embodiments the configuration shown in FIG. 5C is the pre-stressed configuration and the configuration shown in FIG. 5B is the stressed configuration. In such embodiments, the actuation elements are deformed (e.g., stretched) relative to their preferred geometries by moving the flared end portion 515 from the second chamber 564b to the first chamber 564a. Accordingly, the actuation assembly 510 illustrated in FIGS. 5A-5C can include one or more actuators 512 configured to operate under compression (e.g., when FIG. 5B is the unstressed configuration and FIG. 5C is the loaded or stressed configuration), under tension (e.g., when FIG. 5C is the unstressed configuration and FIG. 5B is the loaded or stressed configuration), and/or a combination thereof.

FIGS. 6A and 6B illustrate views of the first actuator 512a of FIG. 5C (e.g., with the flared end portion 515 positioned in the second receiving chamber 564b) with other aspects of the actuation assembly 510 omitted for clarity. In the illustrated embodiment the first and second targets 520a-b are configured generally similar or the same as the targets 120a-b of FIG. 1C, and the second target 520b is additionally configured as a control element, e.g., to control fluid flow. For example, referring first to FIG. 6A the first actuator 512a is in a first position such that the second target 520b does not substantially prevent fluid flow through a first fluid inlet 524a. In some embodiments, the first target 520a can at least partially contact an interior surface of the well 560 when the first actuator 512a is in the first position.

Referring next to FIG. 6B, the first actuator 512a has transitioned to a second position such that the second target 520b substantially prevents fluid flow through the first fluid inlet 524a. Energy (e.g., heat) applied to the first target 520a and/or the first actuation element 518a can cause the first actuation element 518a to transition toward its preferred geometry, which can bend or pivot the actuator body 514 relative to the second receiving chamber 564b and move the second target 520b to contact the first fluid inlet 524a. In at least some embodiments, the second target 520b can at least partially deform (e.g., deflect, bend, pivot, move, etc.) a side or wall 525 of the first fluid inlet 524a such that the wall 525 at least partially obstructs the first fluid inlet 524a, e.g., to substantially prevent fluid flow through the first fluid inlet 524a. To return to the first position, energy can be applied to the second target 520b and/or the second actuation element 518b to cause the second actuation element 518b to transition toward its preferred geometry and deform the first actuation element 518a, as described previously. In embodiments that include the wall 525, the wall 525 can be generally resilient or at least partially resistant to deformation, such that the wall 525 returns to a configuration that does not substantially prevent fluid flow through the first inlet when the first actuator 512a returns to the first position. In some embodiments, the second target 520b moves in a direction that is generally axially aligned with the first fluid inlet 524a, e.g., as described previously with reference to the control element 116 of FIGS. 2A-2C. Accordingly, it is expected that the second target 520b will exhibit the same or generally similar improved sealing performance as the control element 116 of FIGS. 2A-2C.

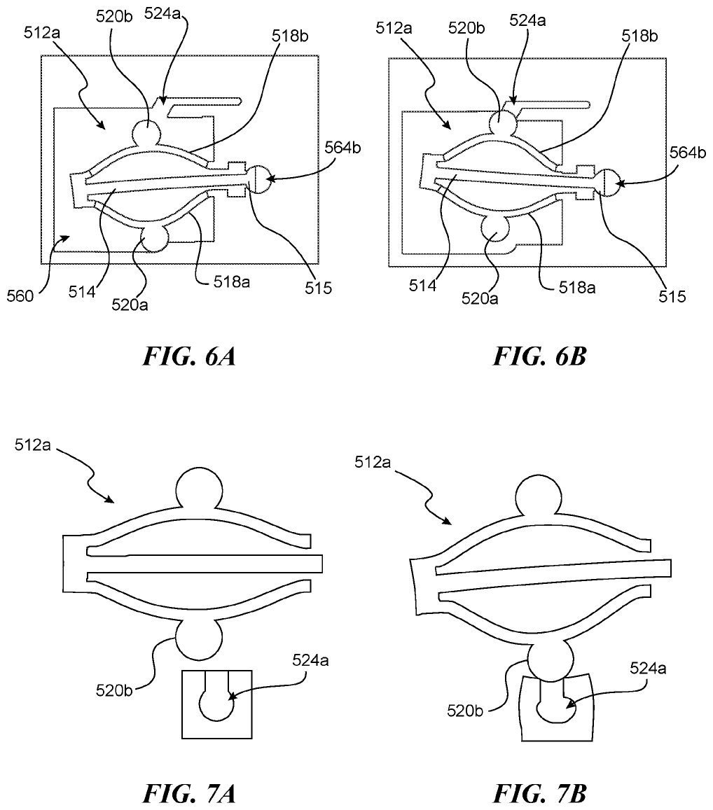

FIGS. 7A and 7B illustrate views of the first actuator 512a of FIGS. 6A and 6B, with certain aspects of the first actuator 512a omitted for clarity. Referring first to FIG. 7A, the first actuator 512a is in a first position such that the second target 520b does not substantially prevent fluid flow through the first fluid inlet 524a. Referring next to FIG. 7B, the first actuator 512a has transitioned to a second position such that the second target 520b contacts the first fluid inlet 524a and substantially prevents fluid flow through the first fluid inlet 524a. In the illustrated embodiments, the first fluid inlet 524a is formed from a flexible and/or elastomeric material such that the second target 520b can at least partially deform the first fluid inlet 524a when in the second position. As discussed previously with reference to the sealing element 340 of FIGS. 3A-3B, it is expected that the deformability of the first fluid inlet 524a will improve the fluidic seal formed between the second target 520b and the fluid inlet 524a when in the second position. The first fluid inlet 524a can be formed from any flexible material, such as an elastomer or any other suitable material capable for forming a substantially fluid seal with the second target 520b.

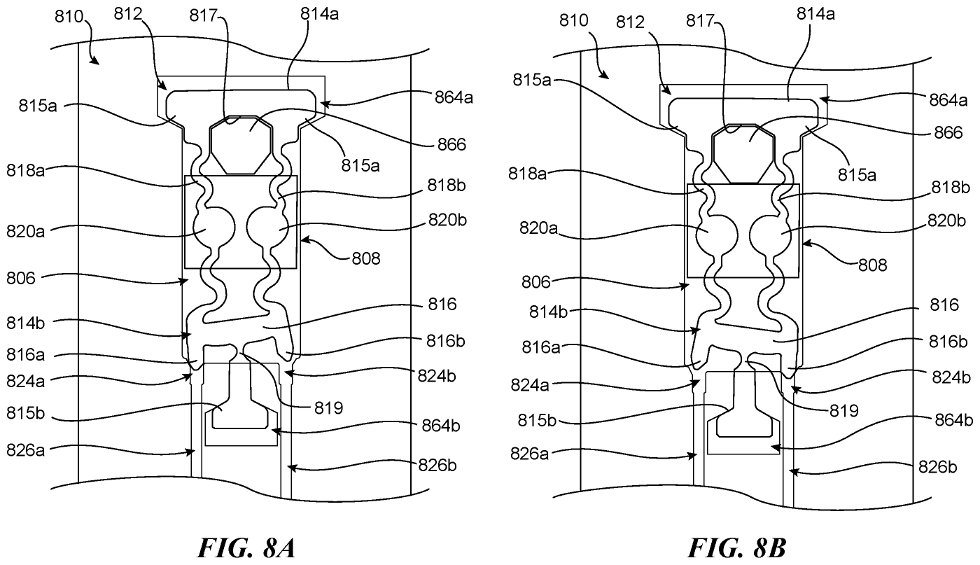

FIGS. 8A and 8B are top views of an actuation assembly 810 configured in accordance with embodiments of the present technology. More specifically, FIG. 8A shows the actuation assembly 810 in a first state or first configuration, and FIG. 8B shows the actuation assembly 810 in a second, different state or configuration. The actuation assembly 810 can include at least some aspects that are generally similar or identical in structure and/or function to the actuation assembly 110 of FIGS. 1C, 2A and 2B, the actuation assembly 310 of FIGS. 3A and 3B, the actuation assembly 410 of FIGS. 4A-4C, and/or the actuation assembly 510 of FIGS. 5A-5C described above. Accordingly, like names and/or reference numbers (e.g., actuation elements 818a-b versus the actuation elements 118a-b, the actuation elements 318a-b, the actuation elements 418a-b, and/or the actuation elements 518a-b) are used to indicate aspects that can be generally similar or identical in structure and/or function.

Referring FIGS. 8A and 8B together, the actuation assembly 810 includes at least one actuator 812 (“the actuator 812”). The actuator 812 can be formed from a single sheet of material, and can include a first body portion 814a, a second body portion 814b, and one or more actuation elements 818 (individually identified as a first actuation element 818a and a second actuation element 818b) extending between the first body portion 814a and the second body portion 814b. In some embodiments, the actuation assembly 810 can be configured to receive fluid (e.g., aqueous) via one or more fluid inlets 808, which can be positioned at least partially between the first body portion 814a and the second body portion 814b or another suitable position. Fluid received via one or more of the fluid inlets 808 can enter a chamber 806. The chamber 806 can include a first chamber portion or region 864a and a second chamber portion or region 864b. In the illustrated embodiment, the first chamber portion 864a and the second chamber portion 85b define opposite ends of the chamber 806. In other embodiments, however, the first and/or second chamber portions 864a-b can have other suitable configurations.

The actuator 812 can be positioned within the chamber 806 and can be configured to control the flow of fluid therethrough. More specifically, the first body portion 814a can be configured to be received within the first chamber portion 864a. In some embodiments, the first body portion 814a includes a first flared end portion 815a configured to contact one or more surfaces of the first chamber portion 864a. Additionally, or alternatively, the first body portion 814a can include an inner retaining surface 817 configured to contact a retaining feature or tab 866 positioned at least partially within and/or proximate to the first chamber portion 864a. In the illustrated embodiment, for example, the retaining surface 817 is positioned between the actuation elements 818a-b and the first body portion 814a includes two first flared end portions 815a, one on both the left and right sides of the retaining surface 817, such that the actuation elements 818a-b are each positioned between one of the corresponding first flared end portions 815a and the retaining surface 817.

The second body portion 814b can include a control element portion 816 and a second flared end portion 815b. The control element portion 816 can include one or more control elements 816a-b (individually identified as a first control element 816a and a second control element 816b). Each of the control elements 816a-b can be configured to control the flow of fluid through one or more channels 826 (individually identified as a first channel 826a and a second channel 826b) by movably interfacing with a corresponding channel opening or fluid inlet 824 (individually identified as a first fluid inlet 824a and a second fluid inlet 824b) of the channels 826. In the illustrated embodiment, for example, each of the control elements 816a-b are configured to be at least partially insertable into the corresponding fluid inlet 824a-b to form a substantially fluid-impermeable seal therewith. In some aspects of the present technology, the control elements 816a-b are expected to form improved seals with the fluid inlets 824 at least because of the motion of the control elements 816a-b relative to the corresponding fluid inlets 824a-b and/or because the control elements 816a-b can be inserted at least partially into the corresponding fluid inlet 824a-b. In some embodiments, one or more sealing elements, such as the sealing element 340 of FIGS. 3A and 3B, can be positioned between the control elements 816a-b and the corresponding fluid inlets 824a-b, e.g., to further improve the seals formed between the control elements 816a-b and the fluid inlets 824a-b. Accordingly, the channels 826 of the actuation assembly 810 are expected to have improved fluid and/or leak resistance when the corresponding control element 816a-b is positioned within (e.g., engages, seals, closes, and/or the like) the associated fluid inlet 824.

In the illustrated embodiment, the control element portion 816 is transitionable between a first position (shown in FIG. 8A) and a second position (shown in FIG. 8B). In these and other embodiments, the control element portion 816 can be configured to transition to one or more other positions, such as a third or intermediate position between the first position and the second position. In the first position (FIG. 8A), the first control element 816a sealingly engages the first fluid inlet 824a to substantially prevent fluid flow therethrough and the second control element 816b is spaced apart from the second fluid inlet 824b to allow fluid flow therethrough. In the second position (FIG. 8B), the first control element 816a is spaced apart from the first fluid inlet 824a to allow fluid flow therethrough, and the second control element 816b sealingly engages the second fluid inlet 824b to substantially prevent fluid flow therethrough. Accordingly, at least one of the fluid inlets 824a-b is expected to be at least partially open to fluid flow whether the control element portion 816 is in the first or second state such that, under a given pressure, the actuation assembly 810 can provide a non-zero flow rate through at least one of the inlets 824a-b. In some embodiments, one or more of the control elements 816a-b do not form a complete fluid seal with the respective fluid inlets 824a-b, but rather permit, for a given pressure, a leakage flow rate, e.g., to ensure that at least some flow through both the channels 826a-b is maintained even when the control elements 816a-b engage are positioned within (e.g., engaging, sealing, closing, and/or the like) the respective fluid inlets 824a-b. In these and other embodiments, the control element portion 816 can be configured to move between one or more intermediate positions between the first position and the second position.

The second flared end portion 815b can be configured to be positioned at least partially within a second chamber portion 864b. In some embodiments, the second body portion 814b can include a joint or pivot feature 819 about which the control elements 816a-b can be pivoted/rotated when the control element portion 816 transitions between the first and second positions. The pivot feature 819 can be positioned between the second flared end portion 815b and the control element portion 816, such that the control element portion 816 can be rotated/pivoted about the pivot feature 819 relative to the second flared end portion 815b. Additionally, in the illustrated embodiment, the pivot feature 819 is positioned between the first and second control elements 816a-b, such that the control element portion 816 is transitionable from the first position (FIG. 8A) to the second position (FIG. 8B) by pivoting the control element portion 816 relative to the second flared end portion 815b to rotate the first and second control elements 816a-b in a clockwise direction about the pivot feature 819. With continued reference to the illustrated embodiment, the control element portion 816 is transitionable from the second position (FIG. 8B) to the first position (FIG. 8A) by pivoting the control element portion 816 relative to the second flared end portion 815b to rotate the first and second control elements 816a-b in a counterclockwise direction about the pivot feature 819. The rotation of the control elements 816a-b about the pivot feature 819 can be driven by actuating the actuation elements 818, as described in detail below.

In some aspects of the present technology, the control element portion 816 can be stable or otherwise generally resistant to movement in both the first and second positions (e.g., “bistable”) at least because one of the control elements 816a-b engages a corresponding one of the fluid inlets 824a-b in both the first and the second positions. While the actuation elements 818 are generally expected to hold the control element portion 816 in the first and/or second position unless/until the actuation elements 818 are actuated (described in detail below), the engagement between the control elements 816a-b and the fluid inlets 824a-b in both the first and second positions is expected to further reduce or prevent unwanted movement of the control element portion 816, such as wiggling or shaking in response to movement of the actuation assembly 810.

The actuation elements 818a-b can generally act in opposition, and each can be actuated to move the control elements 816a-b and transition the control element portion 816 between the first and second positions. In the illustrated embodiment, for example, when the control element portion 816 is in the first position, the second actuation element 818b can be actuated to move the control element portion 816 toward and/or to the second position; when the control element portion 816 is in the second position, the first actuation element 818a can be actuated to move the control element portion 816 toward and/or to the first position. Each of the actuation elements 818a-b can include a respective target 820 (individually identified as a first target 820a and a second target 820b). The targets 820 can extend (e.g., laterally, horizontally, etc.) from the respective actuation elements 818 and can be configured to receive energy (e.g., laser energy) from an energy source external to the patient to selectively and independently actuate the respective actuation elements 818a-b.

The actuation elements 818 can be “stressed,” “strained,” “loaded,” or deformed relative to their preferred geometries by placing the first and second body portions 814a-b in the respective first and second chambers 864a-b. In the illustrated embodiment, for example, the first and second chambers 864a-b are spaced apart such that placing the first body portion 814a in the first chamber portion 864a and the second flared end portion 815b of the second body portion 814b in the second chamber portion 864b can strain or stretch the actuation elements 818 extending therebetween, thereby deforming the actuation elements 818 relative to their preferred/original geometries. The first and second flared ends 815a-b can each be configured to maintain the actuator 812 in this strained/stretched state by, for example, contacting respective surfaces within the corresponding first and second chambers 864a-b which prevent the first and second body portions 814a-b from moving toward each other. Additionally, or alternatively, the retaining surface 817 can be configured to maintain the actuator 812 in the tensioned/stretched state by, for example, contacting the retaining feature 866 to prevent the first and second body portions 814a-b from moving toward each other.

Although the actuation elements 818 in FIGS. 8A and 8B are illustrated as operating under tension (e.g., elongated/strained relative to their preferred geometry), in other embodiments the actuator 812 can be configured to operate under compression, for example, such that the first and second body regions 814a-b can be advanced toward each other to shorten or compress the actuation elements 818 relative to their preferred geometries. Further, although the actuation assembly 810 includes one actuator 812 with two control elements 816a-b that correspond to two fluid inlets 824a-b in the embodiment illustrated in FIGS. 8A and 8B, in other embodiments the actuation assembly 810 can include more actuators 812, individual ones of which can include more or fewer control elements 816a-b and/or fluid inlets 824. In at least some embodiments, the number of control elements 816a-b can equal the number of fluid inlets 824.

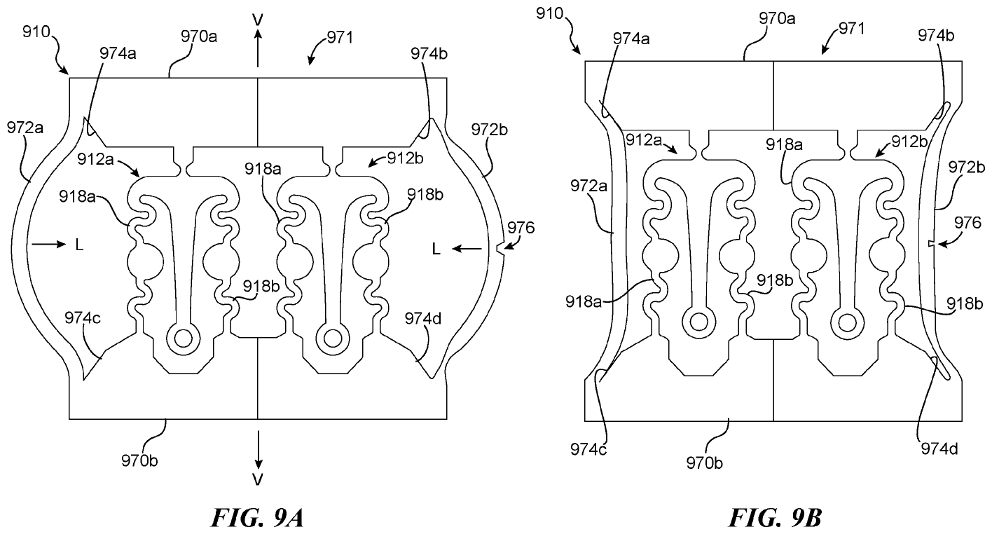

FIGS. 9A and 9B are top views of an actuation assembly 910 configured in accordance with further embodiments of the present technology. More specifically, FIG. 9A shows the actuation assembly 910 in a first state or configuration, and FIG. 9B shows the actuation assembly 910 in a second, different state or configuration. The actuation assembly 910 can include at least some aspects that are generally similar or identical in structure and/or function to the actuation assembly 110 of FIGS. 1C, 2A and 2B, the actuation assembly 310 of FIGS. 3A and 3B, the actuation assembly 410 of FIGS. 4A-4C, the actuation assembly 510 of FIGS. 5A-5C, and/or the actuation assembly 810 of FIGS. 8A and 8B. Accordingly, like names and/or reference numbers (e.g., actuation elements 918a-b versus the actuation elements 118a-b, the actuation elements 318a-b, the actuation elements 418a-b, the actuation elements 518a-b, and/or the actuation elements 818a-b) are used to indicate aspects that can be generally similar or identical in structure and/or function.

Referring FIGS. 9A and 9B together, the actuation assembly 910 can be formed from a single sheet of material and can include one or more body regions 970 (individually identified as a first body region 970a and a second body region 970b), one or more loading or priming arms 972 (individually identified as a first priming arm 972a and a second priming arm 972b), and one or more actuators 912 (individually identified as a first actuator 912a and a second actuator 912b). One or more of the priming arms 972 can include at least one groove or notch 976. Each of the actuators 912 can extend between the first and second body regions 970a-b and include one or more actuation elements 918 (individually identified as a first actuation element 918a and a second actuation element 918b). In the illustrated embodiment, the first priming arm 972a is coupled to a left side of the first and second body regions 970a-b and the second priming arm 972b includes the notch 976 and is coupled to a right side of the first and second body regions 970a-b, such that the body regions 970a-b and the priming arms 972a-b define a priming frame or assembly 971 extending around the actuators 912. In other embodiments, one or both of the priming arms 972 can have a different configuration. In at least some embodiments, for example, the first priming arm 972a can include at least one notch 976 and the second priming arm 972b can be notch-less, or both the first and second priming arms 972a-b can have a same configuration (e.g., both including at least one notch 976, or both notch-less).

The priming frame 971 can be configured to strain/deform the actuation elements 918 relative to their preferred/original geometries. In the illustrated embodiment, for example, the priming arms 972a-b can be bent or deflected (e.g., inwardly, laterally, and/or the like) along a first axis, as indicated by arrows L, from a first position (FIG. 9A) to a second position (FIG. 9B). The bending/deflection of the priming arms 972a-b can thereby cause one or more of the body regions 970a-b to move along a second axis as indicated by arrows V (e.g., outwardly, vertically, and/or the like) and transition the actuation assembly 910 between a first state (FIG. 9A) and a second state (FIG. 9B). When the actuation assembly 910 is in the first state, the actuation elements 918 can be at or near their preferred geometries, as shown in FIG. 9A. When the actuation assembly 910 is in the second state, the actuation elements 918 can be stretched or otherwise deformed relative to their preferred (e.g., as-manufactured) geometries, as shown in FIG. 9B. Additionally, or alternatively, the actuation assembly 910 can be transitioned from the first state (FIG. 9A) to the second state (FIG. 9B) by moving one or more of the body regions 970a-b along the second axis (as indicated by the arrows V) to thereby deform the actuation elements 918 relative to their preferred geometries. Then, one or more of the priming arms 972a-b can be bent/deflected from the first position toward the second position to secure/lock the actuation assembly 910 in the second state and/or at least partially inhibit or prevent the body regions 970a-b from moving back toward the first state.

Each of the priming arms 972a-b can be configured to be stable or otherwise generally resistant to bending/deflection in their respective first and second positions. In at least some embodiments, for example, the priming arms 972 can be configured to lock or snap into the inwardly-deflected second position shown in FIG. 9B in response to movement of the priming arms 972 in the direction indicated by the arrows L (FIG. 9A). The notch 976 in the second priming arm 972b can further improve the stability of the second priming arm 972b as the actuation assembly transitions between the first and second states, for example, by reducing the resistance of the second priming arm 972b to inward deflection.

In the illustrated embodiment, when the actuation assembly 910 is in the first state (FIG. 9A), the priming arms 972 define a first width of the actuation assembly 910 and the body regions 970 define a second width less than the first width. Accordingly, in some embodiments, the actuation assembly 910 can be transitioned from the first state to the second state by positioning the actuation assembly 910 within a chamber or other space having a width generally similar or identical to the second width of the body regions 970, such that the priming arms 972 are deflected inwardly by the chamber from the first position to the second position to thereby drive the body regions 970 apart and transition the actuation assembly 910 from the first state to the second state.

In some embodiments, one or more of the body regions 970 include one or more priming surfaces 974 (individually identified in the illustrated embodiment as a first priming surface 974a and a second priming surface 974b of the first body region 970a, and a third priming surface 974c and a fourth priming surface 974d of the second body region 970b.) One or more of the priming surfaces 974 can be configured to improve the movement of the priming arms 972 and/or the body regions 970 relative to each other, and/or configured to improve strain distribution across one or more portions (e.g., one or both of the body regions 970a-b) of the actuation assembly 910. As best seen in the embodiment illustrated in FIG. 9B, for example, the first priming arm 972a contacts the first and third priming surfaces 974a, 974c when the priming arm 972a is in the second position and/or the actuation assembly 910 is in the second state. The first and third priming surfaces 974a, 974c can be angled or sloped inwardly (e.g., toward the actuators 912) such that the contact between first priming arm 972a and the first and third priming surfaces 974a, 974c can drive the body regions 970a-b away from each other and deform the actuation elements 918 relative to their preferred geometry. Additionally, or alternatively, the contact between the first priming arm 972a and the first and third priming surfaces 974a, 974c can at least partially inhibit or prevent the body regions 970a-b from moving toward each other. Accordingly, one or more of the priming surfaces 974 can reduce, minimize, and/or prevent strain/deformation-induced “recoil” or other motion of the actuation assembly 910 after the actuation assembly 910 has been transitioned toward/to the second state (FIG. 9B). For example, moving the priming arms 972 inwardly toward the respective priming surfaces 974 can increase the stiffness of the actuation assembly 910 and thereby at least partially inhibit or prevent the actuation assembly 910 from returning from the second state (FIG. 9B) toward/to the first state (FIG. 9A). Thus, in some aspects of the present technology, the priming frame 971 and/or the actuation assembly 910 can be stable or otherwise generally resistant to unwanted movement in both the first and second state (e.g., bistable), which is expected to further inhibit or prevent the actuation elements 818 from returning to their preferred geometries unless/until actuated via energy. In these and other embodiments, one or more of the priming surfaces 974 can be configured such that they are not contact by the priming arms 972. In the illustrated embodiment, for example, the second priming arm 972b is spaced apart from (e.g., does not contact) the second and fourth priming surfaces 974b, 974d when the actuation assembly 910 is in the first and second states.

As one skilled in the art will appreciate, any of the actuation assemblies and/or actuators described above can be used with the system 100 and/or another suitable adjustable shunting system to control the flow of fluid therethrough. Moreover, certain features described with respect to one actuation assembly and/or actuator can be added or combined with another actuation assembly and/or actuator. Accordingly, the present technology is not limited to the actuation assemblies and/or actuators expressly identified herein.

The present technology may provide additional advantages beyond those explicitly described herein. For example, the present technology may provide enhanced surface quality for the actuation assemblies and/or shunting systems, better mechanical properties of the actuation assemblies and/or shunting systems, and/or enable a larger selection of materials to be used for fabricating the actuation assemblies and/or shunting systems.

### Examples

Several aspects of the present technology are set forth in the following examples:

1. An actuation assembly for controlling fluid flow through an adjustable shunt, the actuation assembly comprising:
a first shape memory actuation element;
a second shape memory actuation element;
a body region positioned between and separating the first and second shape memory actuation elements, wherein the body region has a lower thermal conductivity than the first and second shape memory actuation elements; and
a control element operably coupled to the first and second shape memory actuation elements,
wherein (i) the first and second shape memory actuation elements are independently actuatable via heat, (ii) when actuated, the first shape memory actuation element is configured to move the control element toward a first position to be substantially free of interference to fluid flow through an aperture of the adjustable shunt, and (iii) when actuated, the second shape memory actuation element is configured to move the control element toward a second position to at least partially cover the aperture.

2. The actuation assembly of example 1 wherein the body region is configured to thermally isolate the first actuation element from the second actuation element.

3. The actuation assembly of example 1 or example 2 wherein the body region has a first mass and the first and second actuation elements each have a second mass, and wherein the first mass is greater than the second mass.

4. The actuation assembly of any of examples 1-3 wherein the first shape memory element and the second shape memory element are arranged in a stacked configuration along a common axis parallel to a central axis of the aperture.

5. The actuation assembly of any examples 1-4 wherein the control element is configured to move between the first and second positions in a plane that is parallel to a center axis extending through the aperture.

6. The actuation assembly of any of examples 1-5 wherein the control element is one of a plurality of control elements, the first and second shape memory actuation elements are a first pair of a plurality of pairs of first and second actuation elements, and the body region is one of a plurality of body regions.

7. The actuation assembly of example 6 wherein the plurality of body regions are formed by a single, unitary structure.

8. An actuation assembly for use with a shunting system, the actuation assembly comprising:
a first sheet including one or more first actuation elements;
a second sheet including one or more second actuation elements;
a third sheet including one or more actuator bodies, wherein each of the one or more actuator bodies have an end region; and
an actuator mount including one or more ports, wherein the one or more ports correspond to and are configured to receive the end regions of the corresponding one or more actuator bodies;
wherein each of the first sheet, the second sheet, the third sheet, and the actuator mount are configured to be combined in a predetermined configuration; and
wherein combining the first sheet, the second sheet, the third sheet, and the actuator mount in the predetermined configuration deforms at least one of the one or more first and second actuation elements relative to their manufactured geometries.

9. The actuation assembly of example 8 wherein:
each of the one or more actuator bodies include a control element positioned opposite the end region;
the third sheet further includes an actuator body support positioned between the control element and the end region and coupling each of the one or more actuator bodies;
the one or more first and second actuation elements have a first length;
the actuator bodies have a second length between the control element and the actuator body support; and
the first length is greater than the second length.

10. The actuation assembly of example 8 or 9 wherein each of the one or more ports correspond to and are configured to receive one of the end regions of the one or more actuator bodies.

11. The actuation assembly of example 8 or 9 wherein at least one of the one or more ports corresponds to and is configured to receive more than one of the end regions of the one or more actuator bodies.

12. The actuation assembly of any of examples 8-11 wherein combining the first sheet, the second sheet, the third sheet, and the actuator mount in the predetermined configuration automatically deforms at least one of the one or more first and second actuation elements.

13. The actuation assembly of any of examples 8-12 wherein combining the first sheet, the second sheet, the third sheet, and the actuator mount in the predetermined configuration simultaneously deforms each of the one or more first and second actuation elements.

14. An actuation assembly for use with a shunting system, the actuation assembly comprising:
a fluid inlet configured to be fluidly coupled to an environment external to the shunting system;
a first actuation element having a first target configured to (i) receive energy from an external energy source, and (ii) disperse the received energy into the first actuation element to drive actuation thereof, wherein the first actuation element is further configured such that, when actuated, the first actuation element moves the first target toward and/or to the fluid inlet to increase a fluid resistance of the fluid inlet; and
a second actuation element, wherein the second actuation element is configured such that, when actuated, the second actuation element moves the first target away from the fluid inlet to decrease the fluid resistance of the fluid inlet.

15. The actuation assembly of example 14 wherein the first target is configured to form a fluid seal with the fluid inlet when the first actuation element moves the first target toward the fluid inlet.

16. The actuation assembly of examples 14 or 15 wherein the fluid inlet is configured to at least partially deform when the first actuation element moves the first target toward the fluid inlet.

17. The actuation assembly of any of examples 14-16 wherein the fluid inlet includes a wall, and wherein the wall is configured to at least partially deform when the first actuation element moves the first target toward the fluid inlet.

18. The actuation assembly of any of examples 14-17, further comprising:
an actuator body having a flared end portion, wherein the first and second actuation elements are coupled to the actuator body;
a first receiving chamber configured to receive the flared end portion and maintain the first and second actuation elements in a first configuration; and
a second receiving chamber configured to receive the flared end portion and cause the first and second actuation elements to be deformed relative to the first configuration.

19. A method for manufacturing an actuation assembly, the method comprising:
forming a first sheet from a first material, wherein the first sheet includes a plurality of first actuation elements;
forming a second sheet from the first material, wherein the second sheet includes a plurality of second actuation elements;
forming a third sheet from a second material, wherein the third sheet includes a plurality of actuator bodies;
forming an actuator mount from a third material; and
combining the first sheet, the second sheet, the third sheet, and the actuator mount in a predetermined configuration to form a plurality of actuators;
wherein combining the first sheet, the second sheet, the third sheet, and the actuator mount in the predetermined configuration deforms the plurality of first and second actuation elements relative to a preferred geometry.

20. A system for selectively controlling fluid flow in a patient, the system comprising:
a drainage element having a channel therethrough and a port in fluid communication with the channel; and
an actuation assembly coupled to the drainage element and configured to control the flow of fluid through the port, the actuation assembly comprising—
   a base plate including a fluid inlet,
   an actuator mount coupled to the actuation assembly,
   an actuator body having a first end region coupled to the actuator mount and a second end region opposite the first end region and including a control element, wherein the control element is aligned with the fluid inlet,
   a first actuation element coupled to the control element, wherein the first actuation element is configured such that, when actuated, the first actuation element pivots the actuator body to move the control element in a first direction toward the fluid inlet, and
   a second actuation element coupled to the control element, wherein the second actuation element is configured such that, when actuated, the second actuation element pivots the actuator body to move the control element in a second direction away from the fluid inlet.

21. The system of example 20 wherein the first and second actuation elements are composed of Nitinol.

22. The system of examples 20 or 21, further comprising a sealing element positioned between the control element and the fluid inlet.

23. The system of any of examples 20-22 wherein:
the first actuation element includes a first target extending from the first actuation element in a first direction, and wherein the first target is configured to receive an input to actuate the first actuation element;
the second actuation element includes a second target extending from the second actuation element in a second direction, and wherein the second target is configured to receive an input to actuate the second actuation element; and
the second direction is different than the first direction.

24. A method for manufacturing an actuation assembly, the method comprising:
forming one or more actuators in a first configuration, wherein in the first configuration—
   each individual actuator of the one or more actuators is positioned in a corresponding well, each corresponding well including a first chamber and a second chamber; and
each individual actuator of the one or more actuators includes a first actuation element, a second actuation element, and an actuator body, the actuator body having a distal end portion residing in the first chamber or the second chamber; and
moving the one or more of the actuators from the first configuration to a second, different configuration in which the distal end portion of the actuator body is residing in the other of the first chamber or the second chamber,
wherein moving the one or more actuators from the first configuration to the second configuration deforms the first and/or second actuation elements relative to a preferred geometry.

25. The method of example 24 wherein:
the distal end portion is positioned in the first chamber when the one or more actuators are in the first configuration;
moving the one or more actuators from the first configuration to the second configuration further includes moving the distal end portion from the first chamber to the second chamber; and
deforming the first and/or second actuation elements includes compressing the first and/or second actuation elements relative to the preferred geometry.

26. The method of example 24 wherein:
the distal end portion is positioned in the second chamber when the one or more actuators are in the first configuration;
moving the one or more actuators from the first configuration to the second configuration further includes moving the distal end portion from the second chamber to the first chamber; and
deforming the first and second actuation elements includes elongating the first and/or second actuation elements relative to the preferred geometry.

27. An actuation assembly for use with a shunting system for selectively controlling fluid flow in a patient, the actuation assembly comprising:
a fluid inlet; and
an actuator configured to selectively control the flow of fluid through the fluid inlet, wherein the actuator includes—
   a first body portion,
   a second body portion including a control element configured to sealingly engage the fluid inlet, and
   an actuation element positioned between the first body region and the second body region,
   wherein the actuation element is configured to transition the control element between (i) a first position in which the control element sealingly engages the fluid inlet and (ii) a second position in which the control element is spaced apart from the fluid inlet to allow fluid flow therethrough.

28. The actuation assembly of example 27 wherein the second body portion further includes a pivot feature, and wherein the actuation element is configured to transition the control element between the first position and the second position by rotating the control element about the pivot feature.

29. The actuation assembly of claim 28 wherein the actuation element is configured to transition the control element between the first position and the second position by rotating the control element about the pivot feature.

30. The actuation assembly of example 28 or example 29 wherein the second body portion further includes a control element portion, wherein the control element extends from the control element portion toward the fluid inlet.

31. The actuation assembly of example 30 wherein the actuation element is configured to transition the control element between the first position and the second position by pivoting the control element portion about the pivot feature.

32. The actuation assembly of any of examples 27-31, further comprising:
a chamber including a first chamber portion and a second chamber portion, wherein—
   the first body portion is configured to be received within the first chamber,
   the second body portion is configured to be received within the second chamber,
   the actuation element is a shape memory actuator having a preferred geometry, and
   the shape memory actuator is deformed relative to the preferred geometry when the first body portion is received within the first chamber and the second body portion is received within the second chamber.

33. The actuation assembly of any of examples 27-32 wherein, in the first position, at least a portion of the control element is positioned within the fluid inlet.

34. The actuation assembly of any of examples 27-33 wherein the actuation element is further configured to transition the control element to a third position between the first position and the second position.

35. The actuation assembly of any of examples 27-34 wherein the inlet is a first inlet and the control element is a first control element, and wherein the actuation assembly further comprises:
a second fluid inlet,
wherein—
   the second body portion further includes a second control element configured to sealingly engage the second fluid inlet,
   in the first position, the first control element sealingly engages the first fluid inlet and the second control element is spaced apart from the second fluid inlet to allow fluid flow therethrough, and
   in the second position, the second control element sealingly engages the second fluid inlet and the first control element is spaced apart from the first fluid inlet to allow fluid flow therethrough.

36. The actuation assembly of any of examples 27-34, further comprising a sealing element positioned between the control element and the fluid inlet and configured to sealingly engage the fluid inlet when the control element is in the first position.

37. An actuation assembly for use with an adjustable shunting system for selectively controlling fluid flow in a patient, the actuation assembly comprising:
a first body region;
a second body region;
an actuator extending between the first body region and the second body region, wherein the actuator includes a shape memory actuation element having an original geometry; and
a pair of priming arms extending between the first body region and the second body region,
wherein the first body region, the second body region, and the pair of priming arms define a priming frame configured to deform the shape memory actuation element relative to the original geometry.

38. The actuation assembly of example 37 wherein the pair of priming arms includes a first priming arm positioned on a first side of the actuator and a second priming arm positioned on a second side of the actuator opposite the first priming arm.

39. The actuation assembly of example 37 or example 38 wherein individual ones of the pair of priming arms are configured to deflect inwardly toward the actuator to drive the first body region away from the second body region and deform the shape memory actuation element relative to the original geometry.

40. The actuation assembly of any of examples 37-39 wherein individual ones of the pair of priming arms are configured to cause movement of the first body region relative to the second body region to transition the priming frame between a first state in which the shape memory actuation element has the original geometry, and a second state in which the shape memory actuation element is deformed relative to the original geometry.

41. The actuation assembly of example 40 wherein individual ones of the pair of priming arms are configured to at least partially prevent the priming frame from returning from the second state toward the first state.

42. The actuation assembly of example 40 or example 41 wherein, when the priming frame is in the second state, individual ones of the pair of priming arms are configured to at least partially prevent the first body region and the second body region from moving toward each other.

43. The actuation assembly of any of examples 40-42 wherein:
in the first state, individual ones of the pair of priming arms have a first position; and
in the second state, individual ones of the pair of priming arms have a second position that is deflected relative to the first position.

44. An actuation assembly for controlling fluid flow through an adjustable shunt, the actuation assembly comprising:
a first shape memory actuation element;
a second shape memory actuation element;
a control element operably coupled to the first and second shape memory actuation elements; and
a sealing element configured to sealingly engage an aperture of the adjustable shunt,
wherein (i) the first and second shape memory actuation elements are independently actuatable via heat, (ii) when actuated, the first shape memory actuation element is configured to move the control element toward a first position to be substantially free of interference to fluid flow through the aperture and in which the sealing element is spaced apart from the aperture to at least partially allow fluid flow therethrough, and (iii) when actuated, the second shape memory actuation element is configured to move the control element toward a second position to cause the sealing element to at least partially prevent fluid flow through the aperture.

45. The actuation assembly of example 44 wherein, in the second position, the control element is configured to press the sealing element against the aperture to form a substantially fluid-impermeable seal therewith.

46. The actuation assembly of example 44 or example 45 wherein, in the first position, the control element is spaced apart from the sealing element and the fluid aperture.

47. The actuation assembly of any of examples 44-46, further comprising an actuator body positioned between the first and second shape memory actuation elements, wherein, when actuated, the first and second shape memory actuation elements are configured to pivot the actuator body to cause the control element to move between the first and second positions.

48. The actuation assembly of any of examples 44-47 wherein the sealing element includes an elastomeric material.

49. The actuation assembly of any of examples 44-48 wherein the sealing element includes at least one of silicone, PDMS, or PMMA.

50. The actuation assembly of any examples 44-49 wherein the control element is configured to move between the first and second positions in a plane that is parallel to a center axis extending through the aperture and the sealing element.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An actuation assembly for use with a shunting system for selectively controlling fluid flow in a patient, the actuation assembly comprising:
a fluid inlet; and
an actuator configured to selectively control the flow of fluid through the fluid inlet, wherein the actuator includes—
a first body portion,
a second body portion including a control element configured to sealingly engage the fluid inlet, and
an actuation element positioned between the first body portion and the second body portion,
wherein the actuation element is configured to transition the control element between (i) a first position in which the control element sealingly engages the fluid inlet and (ii) a second position in which the control element is spaced apart from the fluid inlet to allow fluid flow therethrough.

2. The actuation assembly of claim 1 wherein the second body portion further includes a pivot feature, and wherein the actuation element is configured to transition the control element between the first position and the second position by moving the control element about the pivot feature.

3. The actuation assembly of claim 2 wherein the actuation element is configured to transition the control element between the first position and the second position by rotating the control element about the pivot feature.

4. The actuation assembly of claim 2 wherein the second body portion further includes a control element portion, wherein the control element extends from the control element portion toward the fluid inlet.

5. The actuation assembly of claim 4 wherein the actuation element is configured to transition the control element between the first position and the second position by pivoting the control element portion about the pivot feature.

6. The actuation assembly of claim 1, further comprising:
a chamber including a first chamber portion and a second chamber portion, wherein—
the first body portion is configured to be received within the first chamber,
the second body portion is configured to be received within the second chamber,
the actuation element is a shape memory actuator having a preferred geometry, and
the shape memory actuator is deformed relative to the preferred geometry when the first body portion is received within the first chamber and the second body portion is received within the second chamber.

7. The actuation assembly of claim 1 wherein, in the first position, at least a portion of the control element is positioned within the fluid inlet.

8. The actuation assembly of claim 1 wherein the actuation element is further configured to transition the control element to a third position between the first position and the second position.

9. The actuation assembly of claim 1 wherein the inlet is a first inlet and the control element is a first control element, and wherein the actuation assembly further comprises:
a second fluid inlet,
wherein—
the second body portion further includes a second control element configured to sealingly engage the second fluid inlet,
in the first position, the first control element sealingly engages the first fluid inlet and the second control element is spaced apart from the second fluid inlet to allow fluid flow therethrough, and
in the second position, the second control element sealingly engages the second fluid inlet and the first control element is spaced apart from the first fluid inlet to allow fluid flow therethrough.

10. The actuation assembly of claim 1, further comprising a sealing element positioned between the control element and the fluid inlet and configured to sealingly engage the fluid inlet when the control element is in the first position.

11. An actuation assembly for controlling fluid flow through an adjustable shunt, the actuation assembly comprising:
a first shape memory actuation element;
a second shape memory actuation element;
a control element operably coupled to the first and second shape memory actuation elements; and
a sealing element configured to sealingly engage an aperture of the adjustable shunt,
wherein (i) the first and second shape memory actuation elements are independently actuatable via heat, (ii) when actuated, the first shape memory actuation element is configured to move the control element toward a first position to be substantially free of interference to fluid flow through the aperture and in which the sealing element is spaced apart from the aperture to at least partially allow fluid flow through the aperture, and (iii) when actuated, the second shape memory actuation element is configured to move the control element toward a second position to cause the sealing element to at least partially prevent fluid flow through the aperture.

12. The actuation assembly of claim 11 wherein, in the second position, the control element is configured to press the sealing element against the aperture to form a substantially fluid-impermeable seal therewith.

13. The actuation assembly of claim 11 wherein, in the first position, the control element is spaced apart from the sealing element and the fluid aperture.

14. The actuation assembly of claim 11, further comprising an actuator body positioned between the first and second shape memory actuation elements, wherein, when actuated, the first and second shape memory actuation elements are configured to pivot the actuator body to cause the control element to move between the first and second positions.

15. The actuation assembly of claim 11 wherein the sealing element includes an elastomeric material.

16. The actuation assembly of claim 11 wherein the sealing element includes at least one of silicone, PDMS, or PMMA.

17. The actuation assembly of claim 11 wherein the control element is configured to move between the first and second positions in a plane that is parallel to a center axis extending through the aperture and the sealing element.

18. An actuation assembly for controlling fluid flow through an adjustable shunt, the actuation assembly comprising:
a first shape memory actuation element;
a second shape memory actuation element;
a body region positioned between and separating the first and second shape memory actuation elements, wherein the body region has a lower thermal conductivity than the first and second shape memory actuation elements; and
a control element operably coupled to the first and second shape memory actuation elements,
wherein (i) the first and second shape memory actuation elements are independently actuatable via heat, (ii) when actuated, the first shape memory actuation element is configured to move the control element toward a first position to be substantially free of interference to fluid flow through an aperture of the adjustable shunt, and (iii) when actuated, the second shape memory actuation element is configured to move the control element toward a second position to at least partially cover the aperture.

19. The actuation assembly of claim 18 wherein the body region is configured to thermally isolate the first actuation element from the second actuation element.

20. The actuation assembly of claim 18 wherein the body region has a first mass and the first and second actuation elements each have a second mass, and wherein the first mass is greater than the second mass.

21. The actuation assembly of claim 18 wherein the first shape memory element and the second shape memory element are arranged in a stacked configuration along a common axis parallel to a central axis of the aperture.

22. The actuation assembly of claim 18 wherein the control element is configured to move between the first and second positions in a plane that is parallel to a center axis extending through the aperture.

23. The actuation assembly of claim 18 wherein the control element is one of a plurality of control elements, the first and second shape memory actuation elements are a first pair of a plurality of pairs of first and second actuation elements, and the body region is one of a plurality of body regions.

24. The actuation assembly of claim 23 wherein the plurality of body regions is formed by a single, unitary structure.

25. An actuation assembly for use with a shunting system, the actuation assembly comprising:
a first sheet including one or more first actuation elements;
a second sheet including one or more second actuation elements;
a third sheet including one or more actuator bodies, wherein each of the one or more actuator bodies have an end region; and
an actuator mount including one or more ports, wherein the one or more ports correspond to and are configured to receive the end regions of the corresponding one or more actuator bodies;
wherein each of the first sheet, the second sheet, the third sheet, and the actuator mount are configured to be combined in a predetermined configuration; and
wherein combining the first sheet, the second sheet, the third sheet, and the actuator mount in the predetermined configuration deforms at least one of the one or more first and second actuation elements relative to their manufactured geometries.

26. The actuation assembly of claim 25 wherein:
each of the one or more actuator bodies include a control element positioned opposite the end region;
the third sheet further includes an actuator body support positioned between the control element and the end region and coupling each of the one or more actuator bodies;
the one or more first and second actuation elements have a first length;
the actuator bodies have a second length between the control element and the actuator body support; and
the first length is greater than the second length.

27. The actuation assembly of claim 25 wherein each of the one or more ports correspond to and are configured to receive one of the end regions of the one or more actuator bodies.

28. The actuation assembly of claim 25 wherein at least one of the one or more ports corresponds to and is configured to receive more than one of the end regions of the one or more actuator bodies.

29. The actuation assembly of claim 25 wherein combining the first sheet, the second sheet, the third sheet, and the actuator mount in the predetermined configuration automatically deforms at least one of the one or more first and second actuation elements.

30. The actuation assembly of claim 25 wherein combining the first sheet, the second sheet, the third sheet, and the actuator mount in the predetermined configuration simultaneously deforms each of the one or more first and second actuation elements.

31. An actuation assembly for use with a shunting system, the actuation assembly comprising:
a fluid inlet configured to be fluidly coupled to an environment external to the shunting system;
a first actuation element having a first target configured to (i) receive energy from an external energy source, and (ii) disperse the received energy into the first actuation element to drive actuation thereof, wherein the first actuation element is further configured such that, when actuated, the first actuation element moves the first target toward and/or to the fluid inlet to increase a fluid resistance of the fluid inlet; and
a second actuation element, wherein the second actuation element is configured such that, when actuated, the second actuation element moves the first target away from the fluid inlet to decrease the fluid resistance of the fluid inlet.

32. The actuation assembly of claim 31 wherein the first target is configured to form a fluid seal with the fluid inlet when the first actuation element moves the first target toward the fluid inlet.

33. The actuation assembly of claim 31 wherein the fluid inlet is configured to at least partially deform when the first actuation element moves the first target toward the fluid inlet.

34. The actuation assembly of claim 31 wherein the fluid inlet includes a wall, and wherein the wall is configured to at least partially deform when the first actuation element moves the first target toward the fluid inlet.

35. The actuation assembly of claim 31, further comprising:
an actuator body having a flared end portion, wherein the first and second actuation elements are coupled to the actuator body;
a first receiving chamber configured to receive the flared end portion and maintain the first and second actuation element in a first configuration; and
a second receiving chamber configured to receive the flared end portion and cause the first and second actuation elements to be deformed relative to the first configuration.

36. A method for manufacturing an actuation assembly, the method comprising:
forming a first sheet from a first material, wherein the first sheet includes a plurality of first actuation elements;
forming a second sheet from the first material, wherein the second sheet includes a plurality of second actuation elements;
forming a third sheet from a second material, wherein the third sheet includes a plurality of actuator bodies;
forming an actuator mount from a third material; and
combining the first sheet, the second sheet, the third sheet, and the actuator mount in a predetermined configuration to form a plurality of actuators,
wherein combining the first sheet, the second sheet, the third sheet, and the actuator mount in the predetermined configuration deforms the plurality of first and second actuation elements relative to a preferred geometry.

37. A system for selectively controlling fluid flow in a patient, the system comprising:
a drainage element having a channel therethrough and a port in fluid communication with the channel; and
an actuation assembly coupled to the drainage element and configured to control the flow of fluid through the port, the actuation assembly comprising—
a base plate including a fluid inlet,
an actuator mount coupled to the actuation assembly,
an actuator body having a first end region coupled to the actuator mount and a second end region opposite the first end region and including a control element, wherein the control element is aligned with the fluid inlet,
a first actuation element coupled to the control element, wherein the first actuation element is configured such that, when actuated, the first actuation element pivots the actuator body to move the control element in a first direction toward the fluid inlet, and
a second actuation element coupled to the control element, wherein the second actuation element is configured such that, when actuated, the second actuation element pivots the actuator body to move the control element in a second direction away from the fluid inlet.

38. The system of claim 37 wherein the first and second actuation elements are composed of Nitinol.

39. The system of claim 37, further comprising a sealing element positioned between the control element and the fluid inlet.

40. The system of claim 37 wherein:
the first actuation element includes a first target extending from the first actuation element in a first direction, and wherein the first target is configured to receive an input to actuate the first actuation element;
the second actuation element includes a second target extending from the second actuation element in a second direction, and wherein the second target is configured to receive an input to actuate the second actuation element; and
the second direction is different than the first direction.

41. A method for manufacturing an actuation assembly, the method comprising:
forming one or more actuators in a first configuration, wherein in the first configuration—
each individual actuator of the one or more actuators is positioned in a corresponding well, each corresponding well including a first chamber and a second chamber; and
each individual actuator of the one or more actuators includes a first actuation element, a second actuation element, and an actuator body, the actuator body having a distal end portion residing in the first chamber or the second chamber; and
moving the one or more of the actuators from the first configuration to a second, different configuration in which the distal end portion of the actuator body is residing in the other of the first chamber or the second chamber,
wherein moving the one or more actuators from the first configuration to the second configuration deforms the first and/or second actuation elements relative to a preferred geometry.

42. The method of claim 41 wherein:
the distal end portion is positioned in the first chamber when the one or more actuators are in the first configuration;
moving the one or more actuators from the first configuration to the second configuration further includes moving the distal end portion from the first chamber to the second chamber; and
deforming the first and/or second actuation elements includes compressing the first and/or second actuation elements relative to the preferred geometry.

43. The method of claim 41 wherein:
the distal end portion is positioned in the second chamber when the one or more actuators are in the first configuration;
moving the one or more actuators from the first configuration to the second configuration further includes moving the distal end portion from the second chamber to the first chamber; and
deforming the first and second actuation elements includes elongating the first and/or second actuation elements relative to the preferred geometry.

44. An actuation assembly for use with an adjustable shunting system for selectively controlling fluid flow in a patient, the actuation assembly comprising:
a first body region;
a second body region;
an actuator extending between the first body region and the second body region, wherein the actuator includes a shape memory actuation element having an original geometry; and
a pair of priming arms extending between the first body region and the second body region,
wherein the first body region, the second body region, and the pair of priming arms define a priming frame configured to deform the shape memory actuation element relative to the original geometry.

45. The actuation assembly of claim 44 wherein the pair of priming arms includes a first priming arm positioned on a first side of the actuator and a second priming arm positioned on a second side of the actuator opposite the first priming arm.

46. The actuation assembly of claim 44 wherein individual ones of the pair of priming arms are configured to deflect inwardly toward the actuator to drive the first body region away from the second body region and deform the shape memory actuation element relative to the original geometry.

47. The actuation assembly of claim 44 wherein individual ones of the pair of priming arms are configured to cause movement of the first body region relative to the second body region to transition the priming frame between a first state in which the shape memory actuation element has the original geometry, and a second state in which the shape memory actuation element is deformed relative to the original geometry.

48. The actuation assembly of claim 47 wherein individual ones of the pair of priming arms are configured to at least partially prevent the priming frame from returning from the second state toward the first state.

49. The actuation assembly of claim 47 wherein, when the priming frame is in the second state, individual ones of the pair of priming arms are configured to at least partially prevent the first body region and the second body region from moving toward each other.

50. The actuation assembly of claim 47 wherein:
in the first state, individual ones of the pair of priming arms have a first position; and
in the second state, individual ones of the pair of priming arms have a second position that is deflected relative to the first position.
